(19) United States
(12) Patent Application Publication
(10) Pub. No.: US 2024/0368655 A1
(43) Pub. Date: Nov. 7, 2024

(54) CLICK HANDLE-MODIFIED DEOXY-FLUOROARABINO NUCLEIC ACID AS A SYNTHETIC GENETIC POLYMER CAPABLE OF POST-POLYMERIZATION FUNCTIONALIZATION

(71) Applicant: THE TRUSTEES OF BOSTON COLLEGE, Chestnut Hill, MA (US)

(72) Inventors: Jia Niu, Lexington, MA (US); Kevin B. Wu, Newton, MA (US)

(21) Appl. No.: 18/651,762

(22) Filed: May 1, 2024

Related U.S. Application Data
(60) Provisional application No. 63/499,476, filed on May 1, 2023.

(51) Int. Cl. C12P 19/34; C07H 19/06; C07H 19/10; C12N 9/12; C12N 15/115

(52) U.S. Cl. CPC C12P 19/34; C07H 19/06; C07H 19/10; C12N 9/1252; C12N 15/115; C12Y 207/07007; C12N 2310/16

(57) ABSTRACT

The disclosure provides 5-octa-1,7-diynyluracil 2′-deoxy-2′-fluoroarabino nucleic acid triphosphate. The “click handle”-modified FANA (cmFANA) triphosphate was recognized by Thermococcus gorgonarius (Tgo) DNA polymerase and was efficiently incorporated, along with FANA nucleotide triphosphates comprising the other three canonical nucleobases, in DNA-templated primer extensions that generated full-length products. The resulting cmFANA polymers exhibited excellent resistance to nuclease degradation and underwent efficient click conjugation to azide-functionalized molecules such as carbohydrates. cmFANA polymers show promise as programmable and evolvable synthetic genetic polymers capable of post-polymerization functionalization.

### CROSS-REFERENCE TO RELATED APPLICATIONS

This application claims the benefit under 35 U.S.C. 119(e) of previously filed U.S. Provisional Patent Application Ser. No. 63/499,476, titled: Click Handle-Modified Deoxy-Fluoroarabino Nucleic Acid as a Synthetic Genetic Polymer Capable of Post-Polymerization Functionalization, filed: 1 May 2023, which is incorporated herein by reference in its entirety for all purposes.

### STATEMENT REGARDING FEDERALLY SPONSORED RESEARCH

This invention was made with government support under grant DP2-HG011027 awarded by the National Institutes of Health. The government has certain rights in the invention.

### SUBMISSION OF SEQUENCE LISTING IN ST.26 XML FILE

The instant application contains twenty-six (26) sequences which have been submitted in accordance with the ST.26 XML WIPO standard. Said XML file containing the sequences is entitled BCU0020US2-SEQ.xml and is approximately 27.6 kB in size, the contents of which are hereby incorporated in their entirety by reference.

### BACKGROUND

The functions of natural nucleic acids such as DNA and RNA have transcended genetic information carriers and now encompass affinity reagents, molecular catalysts, nanostructures, data storage, and many others. However, the vulnerability of natural nucleic acids to nuclease-mediated degradation and the lack of chemical functionality have imposed a significant constraint on their applications.

Xeno(biotic) nucleic acids (XNAs) comprising a backbone other than the naturally occurring sugar-phosphate backbone present in DNA or RNA have been developed to minimize nuclease susceptibility. XNAs include bridged nucleic acids (BNAs), cyclohexene nucleic acids (CeNAs), 2′-deoxy-2′-fluoroarabino nucleic acids (FANAs), glycol nucleic acids (GNAs), 1,5-anhydrohexitol nucleic acids (HNAs), locked nucleic acids (LNAs), 2′-O-methyl ribonucleotides, morpholino nucleic acids (MNAs), peptide nucleic acids (PNAs), and threose nucleic acids (TNAs). The majority of XNA structures developed to date incorporate the same nucleobases adenine (A), cytosine (C), guanine (G), and thymine (T) or uracil (U) as the natural nucleic acids. To enable efficient uptake of XNA building blocks in enzymatic polymerization reactions, rational mutagenesis and directed evolution have generated engineered polymerases that have altered substrate specificity and can tolerate unnatural XNA nucleotide triphosphates (NTPs).

Installation of functional groups to the nucleobases has expanded the chemical repertoire and functions of nucleic acids. A variety of hydrophobic groups have been incorporated into DNA and RNA aptamers, including Slow Off-rate Modified Aptamers (SOMAmers) and highly functionalized nucleic acid polymers (HFNAPs). While this approach provides quantitative incorporation of base-modified nucleotides, bulky modifications often become challenging to incorporate and necessitate re-engineering of the polymerases.

An alternative strategy to introduce nucleobase modifications is incorporation of nucleotides comprising click handle-modified bases followed by efficient conjugation of a variety of chemical motifs via click chemistry. The click approach has introduced hydrophobic groups and carbohydrates onto DNA aptamers.

### SUMMARY

Described herein is the synthesis of a “click handle”-modified FANA (cmFANA) triphosphate nucleotides and nucleic acids comprising the same, e.g., 5-octa-1,7-diynyluracil 2′-deoxy-2′-fluoroarabino triphosphate (“C8-alkyne-FANA UTP”, compound 11 in FIG. 2). C8-alkyne-FANA UTP was recognized by Thermococcus gorgonarius (Tgo) DNA polymerase and was efficiently incorporated, along with FANA nucleotide triphosphates comprising the other three canonical nucleobases, in DNA-templated primer extension reactions that generated full-length products. The resulting cmFANA polymers containing C8-extended alkyne groups exhibited excellent resistance to nuclease degradation and underwent efficient click conjugation to azide-functionalized molecules such as carbohydrates. The DNA-display approach enabled the critical genotype-phenotype linkage for amplifying the genetic information of cmFANA polymers, making feasible the further evolution of cmFANA-based aptamers. The cmFANA technology is a valuable platform for presenting a variety of chemical functionalities including carbohydrates, fluorophores, and hydrophobic or charged moieties. In addition, cmFANA polymers are a promising platform for serving as programmable and evolvable synthetic genetic polymers capable of post-polymerization functionalization.

### BRIEF DESCRIPTION OF THE DRAWINGS

A better understanding of features and advantages of the present disclosure will be obtained by reference to the following detailed description, which sets forth illustrative embodiments of the disclosure, and the accompanying drawings.

FIG. 1 shows an overview of the cmFANA technology described herein. FIG. 1(a) illustrates a structural comparison between a DNA polymer and a cmFANA polymer. FIG. 1(b) illustrates a schematic overview of the creation of a cmFANA polymer.

FIG. 2 shows a scheme used to synthesize C8-alkyne-FANA UTP (11).

FIG. 3 shows HPLC traces and ¹H-NMR and ³¹P-NMR spectra of C8-alkyne-FANA UTP. FIG. 3(a) presents the HPLC traces. FIG. 3(b) and FIG. 3(c) present ¹H-NMR, ³¹P-NMR and mass spectrometry characterizations.

FIG. 4 shows that C8-alkyne-FANA UTP successfully served as a substrate for Thermococcus gorgonarius (Tgo) DNA polymerase. Various amounts of the polymerase were added to the reaction. The extended product was detected via a Cy5 fluorescent tag at the 5′-end of the primer.

FIG. 5 demonstrates cmFANA polymer as a functionalizable synthetic genetic polymer for in vivo applications. FIG. 5(a) illustrates conjugation of azido-fluorescein to a cmFANA polymer via CuAAC. FIG. 5(b) shows that a Cy5-labeled, single-stranded DNA oligonucleotide was almost completely degraded after 6 hours of incubation in human serum. FIG. 5(c) shows a fluorescein-labeled cmFANA polymer was completely stable after 24 hours of incubation in human serum (c).

FIG. 6 shows successful click conjugation of azidoethyl-penta-O-acetyl-mannose (Man-N3), 1-azido-lactose (Lac-N3), and 1-azido-2,6-sialyllactose (SA-Lac-N3) to a cmFANA polymer (FIG. 6c, lanes 2-4 in denaturing PAGE, respectively).

FIG. 7 shows DNA-display and native PAGE analysis of a carbohydrate-conjugated cmFANA library. The genetic information of the mannose-conjugated cmFANA library was amplified and reproduced through a complete DNA-display cycle.

FIG. 8 shows mock selection of a carbohydrate-conjugated cmFANA sequence via DNA-display.

FIG. 9 shows the selection cycle for a Con A-binding cmFANA aptamer.

FIG. 10 presents the enrichment from each round of selection determined by qPCR. A series of 10-fold dilution of single sequence (standard samples) along with water as control were subjected to qPCR to obtain the ct (cycle threshold) value, which was defined as the number of cycles required for the SYBR Green fluorescent signal to exceed the set threshold (upper left). A standard curve was plotted based on the ct values of the five standard samples (upper right). Prepared samples of each round of recovered selection sequences after Exo-1 treatment were run alongside the standard samples to obtain the ct values and calculate the recovery rate (bottom table).

FIG. 11 shows that the next-generation sequencing result shows a decrease in sequence diversity. A visualization of the ratio of unique sequences to the total sequence read of each sample pool from original start library, post-selection library of round 1, and post-selection library of round 2 (upper chart). Representation of unique sequences are categorized by ratio: unique sequences with fraction larger than 0.01 are in red; unique sequences with fraction between 0.003 and 0.01 are in orange; unique sequences with fraction between 0.001 and 0.003 are in yellow; unique sequences with fraction between 0.0003 and 0.001 are in green; unique sequences with fraction between 0.0001 and 0.0003 are in light blue; unique sequences with fraction smaller than 0.0001, including singletons are in dark blue; and unique sequences with fraction equals 0, as a control factor, are in grey. Also, the decreasing population of unique sequences after each round indicates the enrichment of certain sequence families that shows lower representation in previous post-selection pool (lower table).

FIG. 12 illustrates that a k-mer matrix was generated from clustered sequences and visualized as a PCA plot where colors correspond to clusters. The two-dimensional k-mer PCA (Principal Component Analysis) plot for the top 6 clusters of second-round post-selection pool shows most cluster families in the random regions with separation from other cluster families. The separations among the clusters reflect their origins from independent, unrelated parent sequences that present in the pool, while the spread of each cluster reflects the sampling of local sequence space around each parent sequence, which results from calculating the point mutations through Levenshtein edit distance (LED) by FASTAptameR 2.0 algorithm.

FIG. 13 illustrates a scheme for generating single-sequence functionally-conjugated cmFAMA aptamer candidates for binding affinity assay.

FIG. 14 demonstrates that the number of functionalities conjugated to cmFANA effects mobility in native gel electrophoresis. (a) reaction scheme for generating single-stranded cmFANA-azidoethyl mannose conjugates. (b) Analysis of gel electrophoresis using 10% TBE PAGE gel with double-stranded DNA-cmFANA hybrid products before and after functionalization via CUAAC “click” reaction. Mobility of each product is consistent with the number of azidoethyl-mannose conjugated to the cmFANA candidate sequences.

FIG. 15 represents chosen aptamer sequences for ConA (Table 2, below) were tested by flow cytometry assay for binding from 0 to nM Alexa Fluor 647 labeled ConA.

FIG. 16 illustrates flow cytometry contour plots indicting binding of single-sequence aptamers to ConA. Single-sequenced aptamers were incubated with 0 nM and 10 nM of fluorescent-labeled ConA to measure the percentage of binding. The vertical red line indicates a cutoff of 1000 au APC fluorescence; numbers indicate the percentage of particles with fluorescence above this threshold. (a) Comparison of aptamer can-1 incubating with 0 nM and 10 nM Con A. (b) Comparison of aptamer can-2 incubating with 0 nM and 10 nM Con A. (c) Comparison of aptamer can-3 incubating with 0 nM and 10 nM Con A. (d) Comparison of aptamer can-4 incubating with 0 nM and 10 nM Con A.

### DETAILED DESCRIPTION

While various embodiments of the present disclosure are described herein, it will be obvious to those skilled in the art that such embodiments are provided by way of example only. Numerous modifications and changes to, and variations and substitutions of, the embodiments described herein will be apparent to those skilled in the art without departing from the disclosure. It is understood that various alternatives to the embodiments described herein may be employed in practicing the disclosure. It is also understood that every embodiment of the disclosure may optionally be combined with any one or more of the other embodiments described herein which are consistent with that embodiment.

Where elements are presented in list format (e.g., in a Markush group), it is understood that each possible subgroup of the elements is also disclosed, and any one or more elements can be removed from the list or group.

It is also understood that, unless clearly indicated to the contrary, in any method described or claimed herein that includes more than one act or step, the order of the acts or steps of the method is not necessarily limited to the order in which the acts or steps of the method are recited, but the disclosure encompasses embodiments in which the order is so limited.

It is further understood that, in general, where an embodiment in the description or the claims is referred to as comprising one or more features, the disclosure also encompasses embodiments that consist of, or consist essentially of, such feature(s).

It is also understood that any embodiment of the disclosure, e.g., any embodiment found within the prior art, can be explicitly excluded from the claims, regardless of whether or not the specific exclusion is recited in the specification.

It is further understood that the present disclosure encompasses analogs, derivatives, salts, solvates, hydrates, clathrates and polymorphs of all of the compounds/substances disclosed herein, as appropriate. The specific recitation of “analogs”, “derivatives”, “salts”, “solvates”, “hydrates”, “clathrates” or “polymorphs” with respect to a compound/substance or a group of compounds/substances in certain instances of the disclosure shall not be interpreted as an intended omission of any of these forms in other instances of the disclosure where the compound/substance or the group of compounds/substances is mentioned without recitation of any of these forms.

It is also understood that the present disclosure encompasses all possible stereoisomers, including all possible diastereomers and enantiomers and racemic mixtures of enantiomers, of the compounds described herein, and not only the specific stereoisomers as indicated by drawn structure or nomenclature. Some embodiments of the disclosure relate to the specific stereoisomers indicated by drawn structure or nomenclature. The specific recitation of the phrase “or stereoisomers thereof” or the like with respect to a compound in certain instances of the disclosure shall not be interpreted as an intended omission of any of the other possible stereoisomers of the compound in other instances of the disclosure where the term “compound” is used without recitation of the phrase “or stereoisomers thereof” or the like.

Headings are included herein for reference and to aid in locating certain sections. Headings are not intended to limit the scope of the embodiments and concepts described in the sections under those headings, and those embodiments and concepts may have applicability in other sections throughout the entire disclosure.

All patent literature and all non-patent literature cited herein are incorporated herein by reference in their entirety to the same extent as if each patent literature or non-patent literature were specifically and individually indicated to be incorporated herein by reference in its entirety.

### Definitions

Unless defined otherwise or clearly indicated otherwise by their use herein, all technical and scientific terms used herein have the same meaning as commonly understood by those of ordinary skill in the art to which this application belongs.

As used in the specification and the appended claims, the indefinite articles “a” and “an” and the definite article “the” can include plural referents as well as singular referents unless specifically stated otherwise or the context clearly indicates otherwise.

The term “exemplary” as used herein means “serving as an example, instance or illustration”. Any embodiment or feature characterized herein as “exemplary” is not necessarily to be construed as preferred or advantageous over other embodiments or features.

The term “about” or “approximately” means an acceptable error for a particular value as determined by one of ordinary skill in the art, which depends in part on how the value is measured or determined. In certain embodiments, the term “about” or “approximately” means within one standard deviation. In some embodiments, when no particular margin of error (e.g., a standard deviation to a mean value given in a chart or table of data) is recited, the term “about” or “approximately” means that range which would encompass the recited value and the range which would be included by rounding up or down to the recited value as well, taking into account significant figures. In certain embodiments, the term “about” or “approximately” means within 10% or 5% of the specified value. Whenever the term “about” or “approximately” precedes the first numerical value in a series of two or more numerical values or in a series of two or more ranges of numerical values, the term “about” or “approximately” applies to each one of the numerical values in that series of numerical values or in that series of ranges of numerical values.

Whenever the term “at least” or “greater than” precedes the first numerical value in a series of two or more numerical values, the term “at least” or “greater than” applies to each one of the numerical values in that series of numerical values.

Whenever the term “no more than” or “less than” precedes the first numerical value in a series of two or more numerical values, the term “no more than” or “less than” applies to each one of the numerical values in that series of numerical values.

The term “compound” encompasses salts, solvates, hydrates, clathrates and polymorphs of that compound. A “solvate” of a compound comprises a stoichiometric or non-stoichiometric amount of a solvent (e.g., water, acetone or an alcohol [e.g., ethanol]) bound non-covalently to the compound. A “hydrate” of a compound comprises a stoichiometric or non-stoichiometric amount of water bound non-covalently to the compound. A “clathrate” of a compound contains molecules of a substance (e.g., a solvent) enclosed in a crystal structure of the compound. A “polymorph” of a compound is a crystalline form of the compound. The specific recitation of “salt”, “solvate”, “hydrate”, “clathrate” or “polymorph” with respect to a compound in certain instances of the disclosure shall not be interpreted as an intended omission of any of these forms in other instances of the disclosure where the term “compound” is used without recitation of any of these forms.

The term “polynucleotide” refers to a polymer composed of nucleotide units. Polynucleotides can contain naturally occurring nucleic acids (e.g., deoxyribonucleic acid [“DNA” ] and ribonucleic acid [“RNA” ]), or/and nucleic acid analogs.

“Aptamers” are short, single-stranded DNA or RNA (ssDNA or ssRNA) molecules that can selectively bind to a specific target, including proteins, peptides, carbohydrates, small molecules, toxins, and even live cells. The term stems from the Latin terms “aptus,” meaning to fit, and “meros,” meaning part. Aptamers assume a variety of shapes due to their tendency to form helices and single-stranded loops. They are extremely versatile and bind targets with high selectivity and specificity. Rather than primary sequence, aptamer binding is determined by formation of tertiary structure. Target recognition and binding involve three-dimensional, shape-dependent interactions as well as hydrophobic interactions, base-stacking, and intercalation. Aptamers bind because they “fit” their targets.

Although aptamers recognize and bind targets of interest just like antibodies, they have several advantages, including: reproducible high affinity & specificity; no or low batch-to-batch variation; increased thermal stability; non-immunogenic; and, lower costs of manufacturing. Canonical nucleic acid (i.e., DNA and RNA)-based aptamers suffer from two significant shortcomings: they are extremely susceptible to nuclease-mediated degradation; and, they lack chemical functionalities often critical to act as highly specific ligands for protein targets.

Aptamers may be created in a fashion in which they may contain one or more nucleic acid or nucleotide analogs. Nucleic acid or nucleotide analogs include without limitation those which have a non-naturally occurring base/nucleobase, have a sugar or non-sugar moiety other than 2′-deoxyribose or ribose, or engage in linkages with additional nucleotides other than the naturally occurring phosphodiester bond, or a combination thereof. Non-limiting examples of nucleic acid or nucleotide analogs include xeno(biotic) nucleic acids (XNAs) having a backbone other than the naturally occurring sugar-phosphate backbone present in DNA or RNA (e.g., bridged nucleic acids [BNAs], cyclohexene nucleic acids [CeNAs], 2′-deoxy-2′-fluoroarabino nucleic acids [FANAs], glycol nucleic acids [GNAs], 1,5-anhydrohexitol nucleic acids [1-NAs], locked nucleic acids [LNAs], 2′-O-methyl ribonucleotides, morpholino nucleic acids [MNAs], peptide nucleic acids [PNAs], and threose nucleic acids [TNAs]), phosphorothioates, phosphorodithioates, phosphorotriesters, phosphoramidates, boranophosphates, methylphosphonates, chiral-methyl phosphonates, and the like. DNA and RNA polynucleotides can be synthesized using a DNA or RNA polymerase or an automated DNA or RNA synthesizer. Polynucleotides containing nucleic acid analogs can be synthesized using, e.g., an engineered DNA or RNA polymerase that recognizes the nucleic acid analogs, a phosphoramidite strategy, or an automated peptide synthesizer in the case of PNAs. The term “nucleic acid molecule” typically refers to a larger polynucleotide. The term “oligonucleotide” typically refers to a shorter polynucleotide. In certain embodiments, an oligonucleotide contains no more than about 50 nucleotides. In some embodiments, when a polynucleotide sequence is represented by a DNA sequence (i.e., A, T, G, C), this also includes the corresponding, or the complementary, RNA sequence (i.e., A, U, G, C) in which “U” replaces “T”.

Conventional notation is used herein to describe polynucleotide sequences: the left-hand end of a single-stranded polynucleotide sequence is the 5′ end; the left-hand direction of a double-stranded polynucleotide sequence is referred to as the 5′ direction. The direction of 5′ to 3′ addition of nucleotides to a newly forming (daughter) DNA strand, or to a nascent RNA transcript, is referred to as the direction of replication or transcription, respectively. The DNA strand having a complementary, antiparallel sequence as a daughter DNA strand or a primary RNA transcript (the “coding strand”) is called the “template strand”. “Upstream” is toward the 5′ end of an RNA molecule, and “downstream” is toward its 3′ end. When considering double-stranded DNA, “upstream” is toward the 5′ end of the coding strand for the gene in question and “downstream” is toward the 3′ end of the coding strand, so the 3′ end of the template strand is upstream of the gene in question and the 5′ end of the template strand is downstream of the gene.

The term “primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as a DNA or RNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize and serves as a site for the initiation of synthesis, but need not reflect the exact sequence of the template. In such a case, specific hybridization of the primer to the template depends on the stringency of the hybridization conditions. A primer can be labeled with an agent that promotes isolation (e.g., biotin), separation (e.g., biotin) or detection (e.g., a fluorescent, chromogenic or radioactive moiety) of the coding strand in single-stranded form or the coding strand hybridized to the template strand.

### Click Handle-Modified 2′-Deoxy-2′-Fluoroarabino Nucleic Acid

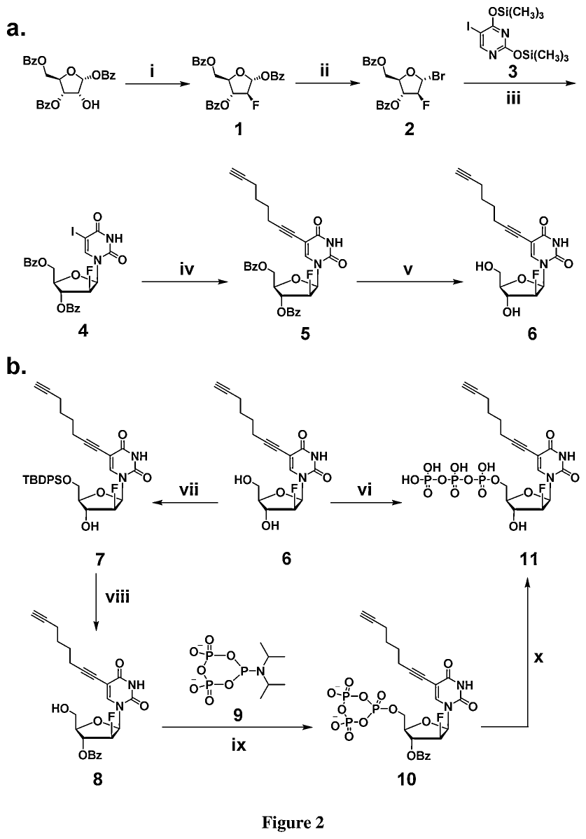

To expand the chemical repertoire of XNA systems and incorporate a wide range of functional side chains, the disclosure describes a click handle-modified FANA (cmFANA) strategy employing 5-octa-1,7-diynyl FANA uracil triphosphate (“C8-alkyne-FANA UTP”, compound 11 in FIG. 2). An alkyne modification at the 5-position of the FANA uridine is used to conjugate to a variety of chemical motifs bearing an azido group via a copper(I)-catalyzed azide-alkyne cycloaddition (CuAAC) reaction. The C8-alkyne-FANA UTP can be efficiently incorporated into cmFANA polymers in DNA-templated primer extension reactions. The high efficiency of the CuAAC reaction enables near quantitative post-polymerization conjugation of various carbohydrate structures ranging from monosaccharides to oligosaccharides that are otherwise challenging to incorporate by engineered polymerases. A DNA-display strategy is used to link the carbohydrate-conjugated cmFANA polymer with its encoding DNA template, laying the foundation for in vitro selection of FANA-based synthetic genetic polymers.

### Representative Embodiments

The following embodiments of the present disclosure are provided by way of illustration and example:

A nucleotide compound designated 5-octa-1,7-diynyluracil 2′-deoxy-2′-fluoroarabino or nucleic acid comprising the same, wherein the nucleotide compound is of the following structure, or a salt, solvate or hydrate thereof:

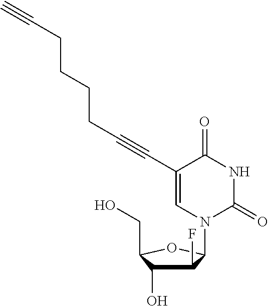

A method of synthesizing 5-octa-1,7-diynyluracil 2′-deoxy-2′-fluoroarabino nucleic acid, or a salt, solvate or hydrate thereof, substantially as described in FIG. 2a.

A compound designated 5-octa-1,7-diynyluracil 2′-deoxy-2′-fluoroarabino nucleic acid triphosphate and having the following structure, or a salt, solvate or hydrate thereof:

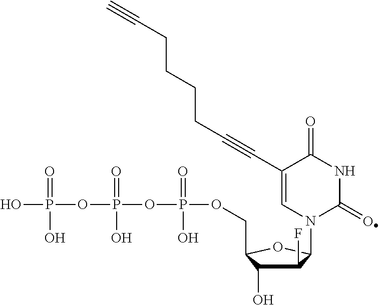

A method of synthesizing 5-octa-1,7-diynyluracil 2′-deoxy-2′-fluoroarabino nucleic acid triphosphate, or a salt, solvate or hydrate thereof, substantially as described in FIG. 2b.

A method of synthesizing an aptamer, comprising mixing a template (e.g., a DNA template), a primer (e.g., a DNA primer), 5-octa-1,7-diynyluracil 2′-deoxy-2′-fluoroarabino nucleic acid triphosphate, and a wild-type or engineered polymerase (e.g., a wild-type or engineered DNA polymerase such as Thermococcus gorgonarius [Tgo] DNA polymerase).

The method of synthesizing an aptamer according to embodiment 5, further comprising mixing 2′-deoxy-2′-fluoroarabino nucleic acid (FANA) analogs of adenosine-5′-triphosphate (ATP), cytidine-5′-triphosphate (CTP) and guanosine-5′-triphosphate (GTP).

The method of synthesizing an aptamer according to embodiment 5 or 6, wherein the primer is labeled with an agent that promotes isolation (e.g., biotin), separation (e.g., biotin) or detection (e.g., a fluorescent or chromogenic compound) of a single-stranded or double-stranded aptamer comprising one or more 5-octa-1,7-diynyluracil 2′-deoxy-2′-fluoroarabino nucleotides.

A method of conjugating a single-stranded or double-stranded aptamer comprising one or more 5-octa-1,7-diynyluracil 2′-deoxy-2′-fluoroarabino nucleotides to an azide-functionalized compound (e.g., a carbohydrate) via a copper(I)-catalyzed azide-alkyne cycloaddition (CuAAC) reaction.

### Representative Examples

The following examples are intended only to illustrate the disclosure. Other procedures, methodologies, techniques, reagents and conditions may alternatively be used as appropriate.

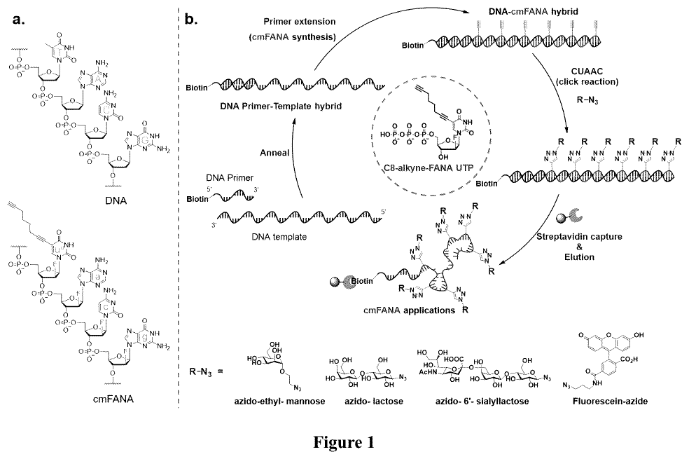

FIG. 1 shows an overview of the cmFANA technology. (a) Structural comparison between a DNA polymer and a cmFANA polymer. (b) Transcription of a DNA template into a cmFANA polymer, conjugation by CuAAC, and the DNA-display strategy to link the cmFANA polymer with its encoding DNA template. The structure of the cmFANA uridine triphosphate, C8-alkyne-FANA UTP, is shown in the middle. Examples of azido-modified compounds conjugated to cmFANA polymers are described below.

### Example 1. Synthesis of C8-alkyne-FANA UTP

FIG. 2 shows a scheme used to synthesize C8-alkyne-FANA UTP (11).
(a) Synthetic route to C8-alkyne-FANA nucleoside 6: i. DAST, DCM, 40° C., 16 hr, 85%; ii. HBr, AcOH, DCM, r.t., 24 hr, 80%; iii. 3, NaI, DCM/ACN, r.t., 7 d, 48%; iv. 1,7-octadiyne, PdCl₂(PPh₃)₂, CuI, Et₃N, DMF, r.t., 16 hr, 76%; v. NaOMe/MeOH, r.t., 2 hr, 84%. (b) Conventional triphosphorylation strategy to C8-alkyne-FANA UTP 11: vi. 1) POCl₃, PO(OMe)₃, 0° C., 8 hr; 2) (NHBu₃)₂H₂P2O₇, Bu₃N, DMF, 0° C., 1.5 hr. An alternative triphosphorylation strategy using a cyclic pyrophosphoryl phosphoramidite (cPyPA) for coupling/ring-opening (steps vii-x) to make 11: vii. TBDPSCl, imidazole, DMF, r.t., 18 hr, 85%; viii. 1) BzCl, pyridine, DCM, 0° C., 2 hr, 2) TBAF, THF, r.t., 1 hr, 90%; ix. 1) 9, 5-(ethylthio)-1H-tetrazole (ETT), MeCN, r.t., 3 hr, 2) mCPBA, 0° C., 5 min; x. 1) D₂O, r.t., 3 hr, 2) NH₄OH, r.t., 18 hr, 60%.

Abbreviations: ACN=acetonitrile; AcOH=acetic acid; Bz=benzoyl; d=day(s); DAST=diethylaminosulfur trifluo ide (Et₂NSF₃); DCM=dichloromethane; DMF=N,N-dimethylformamide; mCPBA=meta-chloroperoxybenzoic acid; r.t.=room temperature; TBAF=tetra-n-butylammonium fluoride; TBDPS=tert-butyldiphenylsilyl; THF=tetrahydrofuran.

In the synthetic scheme shown in FIG. 2, fluorination of commercially available 1,3,5-tri-O-benzoyl-α-(D)-ribofuranose by diethylaminosulfur trifluoride (DAST) gave 2-fluoroarabanose 1. Bromination of compound 1 afforded the glycosyl donor 2, which was subjected to glycosylation with 5-iodouracil to yield a crude product with an a/B isomer ratio of 1:3. The desired β-isomer 4 was isolated by recrystallization. Sonogashira coupling of compound 4 and 1,7-octadiyne furnished 3′,5′-benzoyl-protected nucleoside 5. An excessive amount (12 equiv.) of 1,7-octadiyne was used to ensure mono-functionalization and avoid crosslinking. Deprotection of compound 5 using sodium methoxide yielded the C8-alkyne-FANA uridine 6.

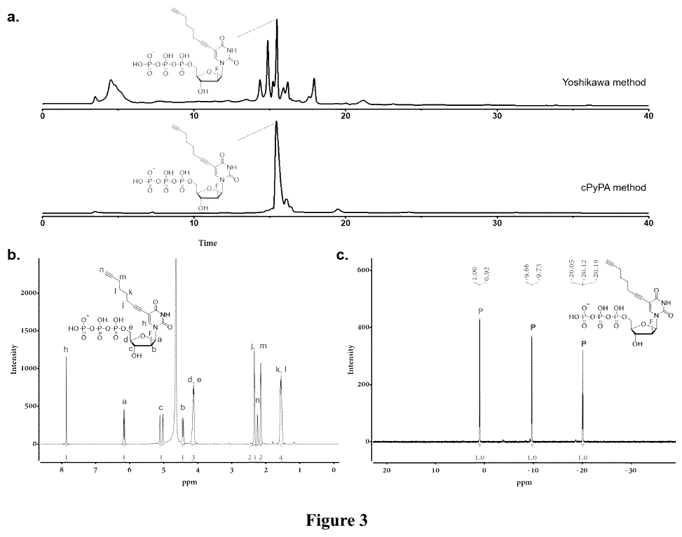

Triphosphorylation of compound 6 was initially attempted using POCl₃ and pyrophosphate following the conventional Yoshikawa phosphorylation protocol (FIG. 2b, vi). This reaction resulted in a complex mixture, which required preparative HPLC for separation, and produced the desired C8-alkyne-FANA UTP 11 in poor yield. The use of cyclic pyrophosphoryl phosphoramidite (cPyPA) 9 provided the desired product 11 in high yield and good purity (FIG. 2b, vii-x). ¹H-NMR, ³¹P-NMR and mass spectrometry characterization confirmed the structure of compound 11 (FIGS. 3b and 3c). Accurate quantitation of compound 11 in the lyophilized product was achieved by ³¹P-NMR using a known amount of the sodium phosphate monobasic salt as the internal standard (data not shown). FIG. 3a shows the HPLC traces of triphosphorylation products using the Yoshikawa method (top) and the cPyPA method (below).

### Example 2. Polymerase Recognition of C8-Alkyne-FANA UTP

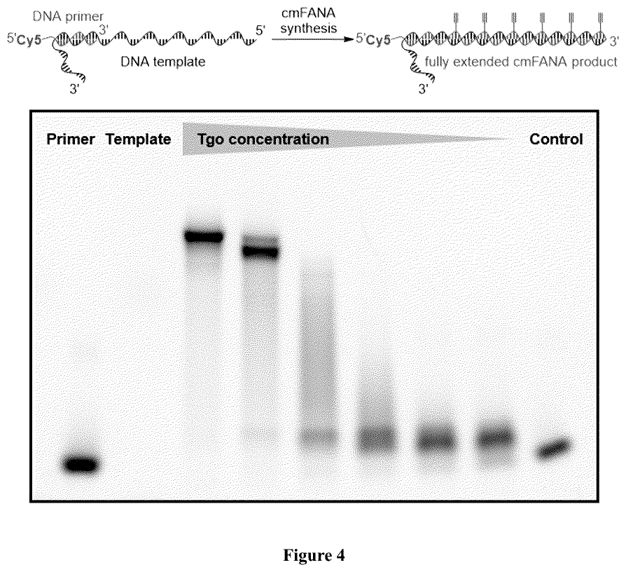

It was hypothesized that Thermococcus gorgonarius (Tgo) DNA polymerase could tolerate the 5-1,7-octadiyne modification on uracil and that the enzyme could incorporate FANA nucleotides. To investigate the hypothesis, a primer extension experiment was performed, in which a DNA primer-template complex was enzymatically extended by Tgo DNA polymerase using commercially available FANA nucleotide triphosphates (A, C, and G) as well as C8-alkyne-FANA UTP 11. The DNA template was prepared to contain a 40-nucleotide (nt) random region to avoid sequence bias, as well as a 24-nt poly-AAC tail at the 3′-end, which would not be transcribed, to provide a mobility difference between extension product (76 nt) and DNA template (100 nt) in denaturing polyacrylamide gel electrophoresis (PAGE). The DNA primer was labeled by a 5′-Cy5 fluorophore to allow easy detection of the extended product. A control experiment in which all FANA nucleotide triphosphates comprising natural bases (A, C, G and T) was also conducted to determine the impact of the 5-1,7-octadiyne modification of uracil on the primer extension reactivity. Tgo DNA polymerase was added in various amounts to confirm that the generation of the extended products was mediated by the Tgo DNA polymerase in a dose-dependent fashion. Both the primer extension reaction involving FANA NTPs of all natural bases and the one involving the FANA ATP, CTP, GTP, and compound 11 produced the full-length products in a polymerase dose-dependent fashion (FIG. 4).

To unambiguously confirm the primer extension product, a cmFANA sequence was synthesized using a DNA template (T-ConA-XL, 98 nt including an 18-nt non-transcribed AAC repeating sequence at the 3′-end) encoding an aptamer targeting Concanavalin A (ConA), and a 5′-biotinylated DNA primer. After the primer extension reaction, the DNA-cmFANA heteroduplex was captured by streptavidin-coated magnetic particles, and the cmFANA polymer was separated from the DNA template strand by treating the captured heteroduplex with sodium hydroxide. The cmFANA polymer was then eluted from the magnetic particles by incubation with concentrated ammonium hydroxide for two hours. Electrospray ionization mass spectrometry (ESI-MS) analysis of the strand-separated cmFANA polymer suggested that the observed molecular weight of the cmFANA polymer (27,632.5 Da) was consistent with the expected value (27,632.5 Da). Taken together, these results indicated that the 5-1,7-octadiyne modification of uracil was well-tolerated by Tgo DNA polymerase and that C8-alkyne-FANA UTP could be quantitatively incorporated in a DNA-templated primer extension reaction mediated by this enzyme.

### Example 3. cmFANA Polymer Exhibited Superior Nuclease Resistance Compared to DNA

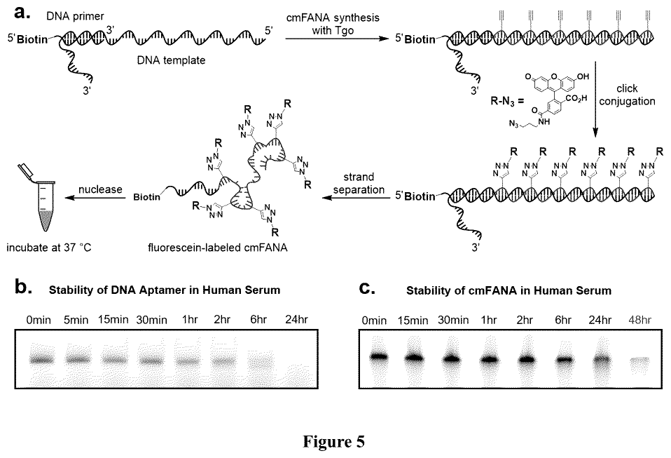

To demonstrate cmFANA polymer as a functionalizable synthetic genetic polymer for in vivo applications, its stability in human serum, which contains a variety of nucleases and mimics the typical environment for in vivo applications, was evaluated. To allow facile quantitation of the cmFANA polymer after human serum treatment, azido-fluorescein was conjugated to the cmFANA polymer via CuAAC (FIG. 5a). A single-stranded DNA oligonucleotide fluorescently labeled by the Cy5 dye was used as a control. Both the DNA oligonucleotide and the cmFANA polymer were incubated in human serum for up to 48 hours, and the remaining nucleic acid species were analyzed by denaturing PAGE. While the DNA oligonucleotide started to degrade after one hour and was almost fully degraded after six hours (FIG. 5b), the cmFANA polymer was completely stable after 24 hours of incubation in human serum and could last even longer than 48 hours (FIG. 5c). The short ssDNA primer sequence that remained at the 5′-end of the cmFANA polymer seemed to withstand degradation as long as the cmFANA region, as the mobility of the product did not change throughout the incubation time. The superior nuclease resistance of cmFANA polymer suggests that it is a promising synthetic genetic polymer for in vivo applications.

### Example 4. Click Conjugation of Carbohydrates to cmFANA Polymer

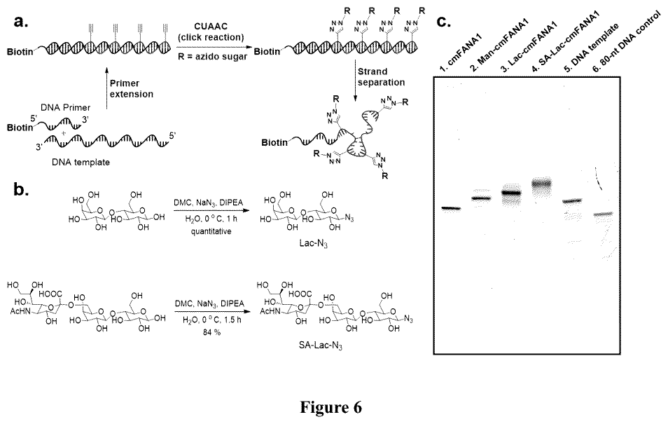

Carbohydrate-modified nucleic acids have emerged as a promising technology to develop affinity reagents for glycan-binding proteins (GBPs) and vaccine epitopes. To demonstrate that cmFANA could serve as a general platform for carbohydrate-modified XNAs, click conjugation of azido-functionalized glycans with cmFANA polymer was evaluated (FIG. 6a). A cmFANA sequence (cmFANA1) was generated from the primer extension reaction using the DNA template T-ConA-XL (98 nt) and a 5′-biotinylated primer. The DNA-cmFANA1 heteroduplex was then coupled with a commercially available monosaccharide azidoethyl-penta-O-acetyl-mannose (Man-N3) by CuAAC. The conjugate was subsequently captured by streptavidin-coated magnetic particles, strand-separated by sodium hydroxide treatment, and deprotected and eluted from the magnetic particles by incubation with concentrated ammonium hydroxide for two hours. Denaturing PAGE analysis confirmed successful click conjugation with Man-N3, in which a clear reduction of the mobility of the band was observed after the click reaction (FIG. 6c, lane 2). ESI-MS analysis of the product of the conjugation reaction suggested quantitative coupling of the mannose residue to all the alkyne groups of cmFANA1 (data not shown). Notably, click conjugation of Man-N3 to cmFANA2, which had the same nucleotide composition as cmFANA1 but was arranged to contain a string of consecutive alkyne groups in the sequence, was not less efficient (data not shown).

Click conjugation of a disaccharide lactose and a trisaccharide 2,6-sialyllactose to cmFANA1 was investigated next. Lactose and commercially available 2,6-sialyllactose were equipped with an azido group at the reducing end by reaction with 2-chloro-1,3-dimethylimidazolinium chloride (DMC) and sodium azide in water (FIG. 6b). The resulting 1-azido-lactose (Lac-N3) and 1-azido-2,6-sialyllactose (SA-Lac-N3) were click-conjugated to the biotinylated DNA-cmFANA heteroduplex, and then strand separation was performed using streptavidin-coated magnetic particles as described above. Denaturing PAGE analysis showed one major band in each lane, which indicated a single major product in each conjugation (FIG. 6c, lanes 3 and 4). While all the cmFANA1 and carbohydrate-cmFANA1 conjugates contained a cmFANA backbone with a length of 80 nt, notable differences in gel mobility of the bands were observed, which was attributed to the different molecular weights of conjugated carbohydrate substrates. ESI-MS analysis suggested that the fully conjugated cmFANA1 with all alkyne groups functionalized by Lac-N3 or SA-Lac-N3 was the major product, although byproducts with 1-3 unreacted alkyne groups were also observed (data not shown). The lower conjugation efficiency of Lac-N3 and SA-Lac-N3 compared to Man-N3 may have been due to the larger size of the carbohydrate substrates. Trace amounts of the cmFANA1-DNA template hybrids resulting from incomplete strand separation were observed above the major band in lanes 2-4.

### Example 5. A DNA-Display Approach to Link a Carbohydrate-Conjugated cmFANA Polymer to its Encoding DNA Template

Directed evolution of modified nucleic acids has become a powerful approach to discover affinity reagents and catalysts with enhanced functions. A critical step in the laboratory evolution process is amplification of the genetic information carried by the evolving population. Amplification is often challenging with base-modified nucleic acids due to an inability to be reverse-transcribed back into native DNA. The bulky carbohydrate-conjugated cmFANA structure raises the question of whether it could be directly reverse-transcribed back to DNA. A series of polymerases, including Taq, Bst LF*, Bst2.0, Bst3.0, Kod, Deep Vent, Tgo and Q5, was tested for their reverse transcriptase activity with cmFANA1 and Man-N3-conjugated cmFANA1. None of the polymerases tested could reverse-transcribe cmFANA1 or Man-N3-conjugated cmFANA1 (data not shown).

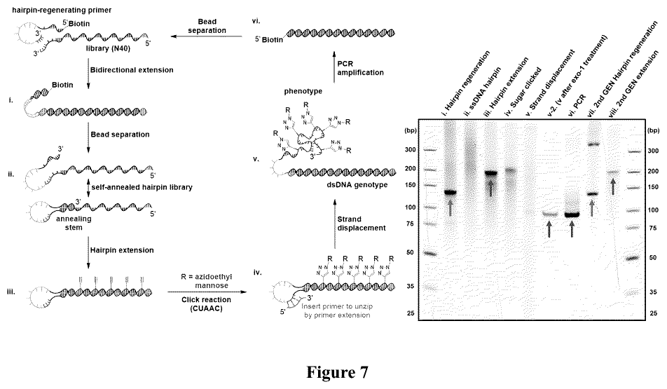

To circumvent the challenge of reverse transcription, an alternative strategy for amplifying the genetic information of a carbohydrate-conjugated cmFANA polymer based on DNA-display was devised. The DNA-display strategy was based on that developed by Temme and Krauss, Curr. Protoc. Chem. Biol., 7:73-92 (2015) in their selection with modified aptamers (SELMA) method, which involves using a hairpin structure to establish the critical phenotype-genotype linkage (FIG. 7). To validate that the genetic information of a library of diverse carbohydrate-conjugated cmFANA sequences could be amplified using DNA-display, the cycle started from a hairpin regeneration process where a biotinylated hairpin-regenerating primer annealed to a library template (94nt, with N40 random region) and was subsequently extended into a double-stranded DNA library (134 bp) through bidirectional extension (FIG. 7, lane i). Streptavidin magnetic particle-enabled strand separation removed the unwanted biotinylated strand, yielding a single-stranded DNA template with a 40nt random region (the genotype) which self-hybridized into a hairpin structure. Due to the presence of the random region, this library appeared as a smear on the native PAGE (FIG. 7, lane ii). Next, Tgo DNA polymerase extended the hairpin from the 3′-end in the presence of FANA ATP, CTP, GTP, and C8-alkyne-FANA UTP 11 (FIG. 7, lane iii), which was followed by conjugation of Man-N3 to the cmFANA library via CuAAC (FIG. 7, lane iv). Next, strand displacement was effected by the addition of a primer that annealed at the loop region and was extended by DNA dNTPs using Bst2.0 WarmStart DNA polymerase. This primer extension step formed a dsDNA and displaced the Man-N3-conjugated cmFANA polymer (the phenotype, hereinafter Man-cmFANA) from the DNA template (FIG. 7, lane v). After strand displacement, the Man-cmFANA-dsDNA hybrid structure was confirmed by exonuclease I digestion, which cleaved the 3′-terminal ssDNA portion along with the displaced Man-cmFANA phenotype, leaving the genotype dsDNA intact (FIG. 7, lane v-2). The genotype dsDNA contained a copy of the same sequence of the Man-cmFANA phenotype in the native DNA form, which could be readily amplified by PCR using a 5′-biotinylated primer. The PCR product was confirmed to have the same length (94 bp) as the genotype dsDNA (FIG. 7, lane vi, compared to lane v-2). Finally, capture of the PCR product by streptavidin-coated magnetic particles and then strand separation yielded a single-stranded DNA coding sequence, which was subjected to the bidirectional extension step again along with the hairpin primer (FIG. 7, lane vii). The regenerated DNA template could be extended once again to produce a second-generation cmFANA polymer library (FIG. 7, lane viii, which is comparable to lane iii). Next-generation sequencing of the initial and regenerated DNA templates indicated similar numbers of unique sequences in the library region, suggesting efficient recovery of the library diversity and that no significant sequence bias was introduced during the DNA-display cycle (data not shown).

In FIG. 7, products of the same mobility are indicated by red, green and purple arrows, with each color labeling a pair of products of the same mobility. Thus, lanes i. and vii. (green); iii. and vii. (red); and, v-2. and vi. (purple) are paired products of the same mobility. A byproduct was observed above the desired product in lane vii after the template regeneration step, which was attributed to over-extension in the bidirectional extension. The amount of this byproduct was greatly reduced after the subsequent strand separation step and did not affect the second round of cmFANA synthesis (lane viii).

### Example 6. Mock Selection of a Carbohydrate-Conjugated cmFANA Sequence Via DNA-Display

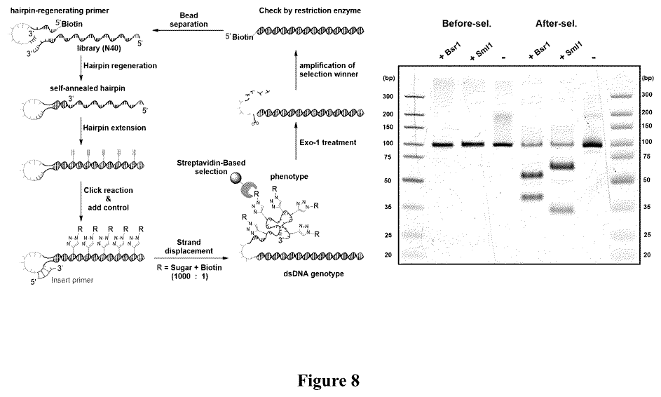

The feasibility of carbohydrate-conjugated cmFANA polymers to be evolved by in vitro selection was validated by a mock selection experiment (FIG. 8). Transcription of a positive control DNA template TConA-Hairpin into a cmFANA sequence followed by conjugation of the resulting product to an azide-functionalized biotin via CuAAC formed a biotinylated cmFANA-DNA hybrid structure. This biotinylated positive control was then added to a Man-cmFANA-DNA hybrid library at a ratio of library:control=1000:1. The selection began with a strand displacement to form the dsDNA genotype and the conjugated cmFANA phenotype. Following capture by streptavidin-coated magnetic particles and six cycles of buffer wash, the dsDNA genotype was eluted by exonuclease I treatment and then was subjected to PCR amplification. The enrichment of the positive control sequence after a full cycle of mock selection was confirmed by restriction enzyme digestion and next-generation sequencing. The restriction enzymes BsrI and SmlI have distinct cut sites and were employed to independently cut the positive control sequence, but not the library, in the coding region before and after the mock selection. Independent digestion by BsrI and SmlI showed that the positive control had become the predominant constituent of the post-selection library. Moreover, next-generation sequencing of the post-selection library indicated that 55% of the total reads had become the positive control sequence. Collectively, these results support the potential of the DNA-display strategy to be applied to the in vitro selection of functionality-conjugated cmFANA sequences.

### Directed Evolution of Multivalent Ligand-Presenting Synthetic Nucleic Acid Affinity Reagents

The first portion of this disclosure described the development of the essential building block for cmFANA and the synthesis of the cmFANA oligonucleotide. Attention is now turned to the evolution and application of cmFANA as a sugar-presenting affinity reagent that targets disease-related carbohydrate binding proteins (“CBPs.”). Embodiments of the disclosure below described present an XNA structure that goes beyond established impressions of nucleic acids and displays a high potential ability as a versatile platform technology.

As above discussed, aptamers based on canonical nucleic acids suffer from extreme susceptibility to nuclease-mediated degradation and the lack chemical functionalities that are frequently critical to act as highly specific ligands for protein targets. Embodiments of the disclosure below discussed seek to apply the cmFANA clickable platform technology above disclosed to develop an unnatural nucleic acid affinity reagent through Darwinian evolution systems, as a demonstration of one of the potential applications of cmFANA platform.

The development of an evolvable cmFANA affinity reagent started from the design of an in vitro selection method that circumvent direct reverse-transcription of selected ligand-conjugated cmFANA genetic polymer. Following the DNA-display strategy described above in Example 5, where the carbohydrate-conjugated cmFANA polymer was covalently linked with its single-sequence encoding DNA template, the feasibility of using a DNA template that was encoded with random library sequence is demonstrated. A mock selection was then practiced with successful enrichment of known single-sequence product out from a library pool mixture. The in vitro selection using Concanavalin A (ConA) as CBP target was carried out with three selection cycles and the enriched sequences were analyzed by next-generation high throughput sequencing. Finally, four aptamer candidates were synthesized and functionalized, ready for further binding affinity assays, paving the way for the development of a simple and powerful screening and characterization method for cmFANA-based aptamer.

Embodiments of the disclosure presented in further detail below demonstrate that: First, the genetic information of a majorly structurally modified aptamer was able to be recovered and amplified using the developed in vitro selection cycle. Next, a cmFANA library successfully underwent three rounds of selection and showed obvious enrichment of the target-binding aptamer population. Finally, aptamer candidates from top enriched cluster families were picked and their single-sequenced cmFANA aptamer structures were generated for preliminary binding measurements using flow cytometry.

For the example embodiments disclosed, a bead-based fluorescent assay using a flow cytometer may be performed using the single-stranded single-sequenced cmFANA aptamers below presented to further study the preliminary binding assay and measured the Kd value of the selected aptamers. Furthermore, using the generated cmFANA aptamers, a surface plasmon resonance (SPR) instrument may also be performed for a more comprehensive study of the binding affinity of the aptamer to a protein target.

Additionally, in view of promising binding affinity results presented for an embodiment example using aptamer can-1 shown via a bead-based fluorescent binding assay, more of the 5′-biotinylated aptamer can-1 with and without mannose sugar conjugation, as well as 5′-biotinylated can-1-scrambled single-stranded sequence, which contains a sequence scramble version of can-1 at the N40 cmFANA region, for future Streptavidin-based aptamer immobilizing binding affinity assay using SPR were prepared. All in all, the cmFANA technology demonstrated represents a valuable sequence-defined biopolymer material that possesses the potential to replace the applications of natural genetic polymers, including the ability to undergo laboratory evolution for the generation of a nuclease-resistant, functionality-conjugatable, and target-specific affinity reagent.

In Vitro Selection of Mannose-Conjugated cmFANA Against Con A

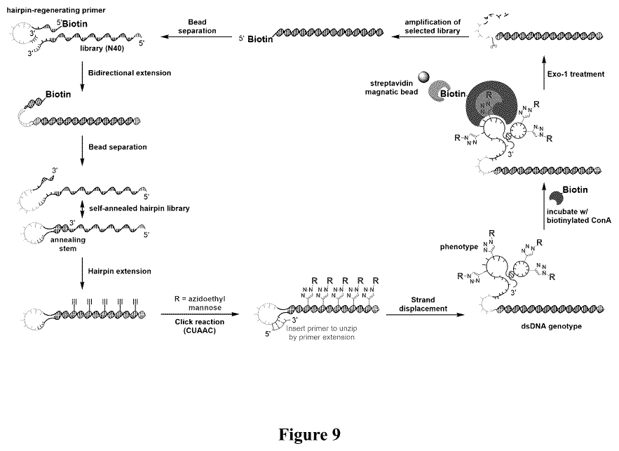

In vitro selection of a functionality-conjugated cmFANA library demonstrated successful recovery and enrichment of the genetic information of functionality-conjugated cmFANA sequences that bind to target protein. To begin, the lectin concanavalin A (Con A) was chosen for its commercial availability and easy access to various Con A conjugates. Biotinylated Con A protein was first tested as the target protein for the selection cycle. After generation of the single-stranded DNA template (with N40 random region, the genotype) that would self-hybridized into a hairpin structure, cmFANA was synthesized by Tgo, by extending the hairpin from the 3′-end to obtain a self-hybridized DNA template-cmFANA double-stranded hairpin structure (FIG. 9). Next, since Con A is known to bind to mannose, 2-azidoethyl-α-D-mannose was conjugated to the cmFANA strand as natural mannose sugar mimetic and Con A specific ligand in this experiment. The functionality-conjugated cmFANA strand was then displaced from the DNA genotype by strand displacement process and allowed to fold into corresponding phenotype.

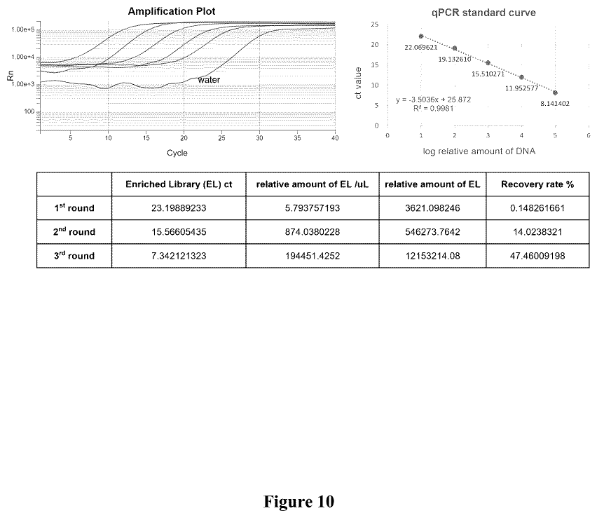

After incubation of the strand-displaced library products with biotinylated Con A, following capture by Streptavidin magnetic particles and ten cycles of buffer wash, the dsDNA genotype was eluted by the Exonuclease-I treatment and was subjected to PCR amplification, strand separation, and hairpin regeneration. Three rounds of selection cycle were performed, which was determined by analyzing the enrichment of recovered post-selection library through qPCR. In a surprising outcome, even functionalized with the Con A-specific ligand, the recovery rate was only around 0.14% for the first round of selection. However, there was then observed a nearly 100-fold enrichment after the second round, and a final 47% recovery rate for the third round (FIG. 10).

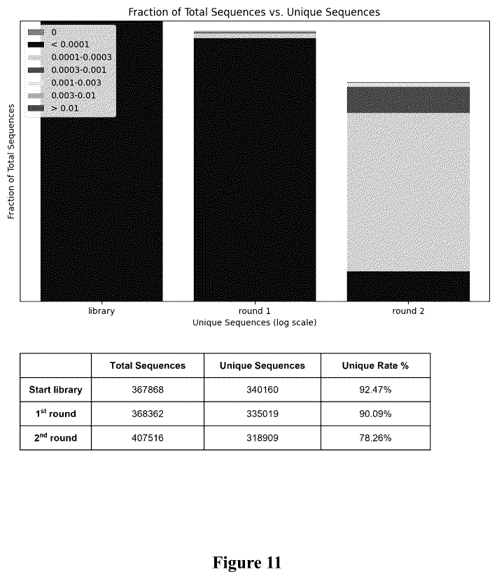

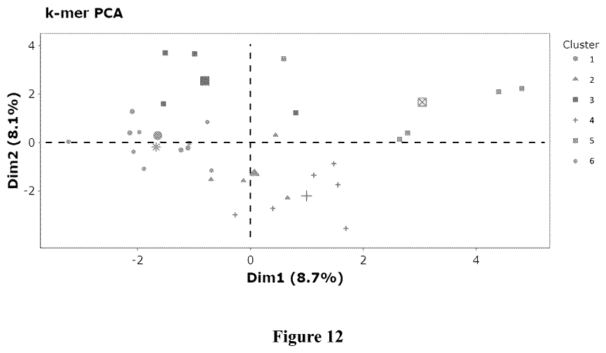

The starting library as well as post-selection libraries from the first and second round of selection cycle were sent for next-generation sequencing. The FASTQ sequencing data was preprocessed using Python and analyzed using FASTAptameR2.0 to count and rank the unique sequences in each pool. As the selection cycle progressed, the population in unique sequence decreased after each round while total reads increased, which shows consistency with the recovery rate determined by qPCR, indicating the enrichment of the representation of specific post-selection unique sequences and overall lower diversity (FIG. 11). Furthermore, four top aptamer cluster families were identified based on sequence relationship, as many of the unique sequences fell into one of these clusters, differing only by little point-mutations (FIG. 12 and Table 1). Four candidate aptamer sequences were picked from each different cluster families for

TABLE 1
Parent sequences of top cluster families.

| | Genotype random region | Total |
|---|---|---|
| Cluster | only (40 nt) | reads |
| 1 | TCGAGATCTTAGGCATCTTTGA | 617 |
| | CGATGACTTCGTCTAACA | |
| 2 | TACGACCCCAATCCATCGTAGA | 153 |
| | ACTATCCCGAATCCTTGT | |
| 3 | GTCAAAGGCACGTGTTGAAACC | 101 |
| | ATATTACTAAGGCACACG | |
| 4 | AGTGATATGCTCAGAAATATCA | 88 |
| | TTCTGGTCATAAATAAAT | |
| 5 | ATACCCAAGACCTACATTATAG | 56 |
| | CAGCGACGATAATTAATG | |
| 6 | CAAAATTTTCAGTTCGACCGTT | 59 |
| | CAACTTCAACTACAAACA | |

The most enriched sequences of each cluster families are listed.
Four parent sequences of each cluster families 1 to 4 were picked for binding affinity assay.
Clusters 1-6 represent SEQ. ID. Nos: 1-6.

### Generating Single-Sequenced Mannose-cmFANA Aptamer Candidates

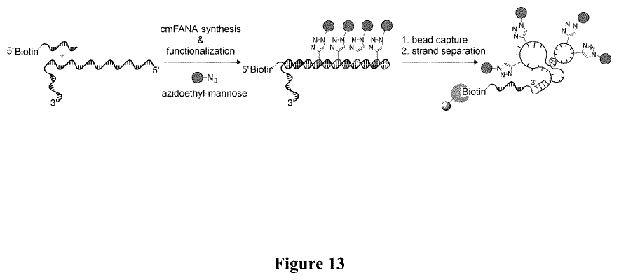

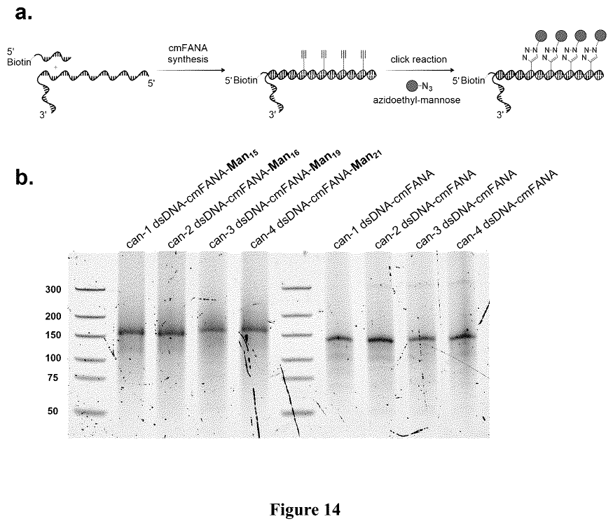

Single-sequenced aptamer candidates were generated by Tgo polymerase conducted primer extension using 5′-biotinylated DNA primer and DNA template with extending region (100 nt template, with 80 nt single-stranded DNA-cmFANA product), as well as FANA nucleotide triphosphates (A, C, and G) and C8-alkyne-FANA UTP. After functionalization with 2-azidoethyl-α-D-mannose, the 5′-biotinylated single-stranded functionality-conjugated cmFANA aptamer product was isolated by immobilization with Streptavidin magnetic bead and then incubation with 250 mM sodium hydroxide to remove DNA template strand (FIG. 13). Native gel analysis showed slower overall mobility for the mannose functionalized cmFANA, compared with non-functionalized cmFANA synthesis products (FIG. 14). Among the functionalized cmFANA products, can-4, conjugated with 21 azidoethyl-mannose, showed slowest electrophoresis mobility, while can-1 and can-2 runs faster with the lowest azidoethyl-mannose conjugations (15 and 16 conjugations, respectively). The generated single-stranded, single-sequenced, and functionality-conjugated cmFANA aptamers are consequently ready for downstream applications and assays.

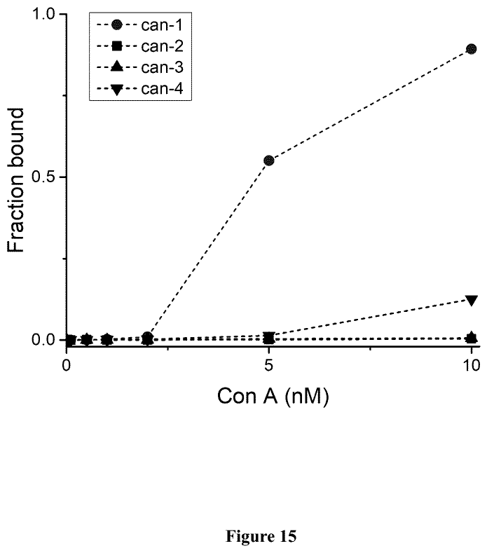

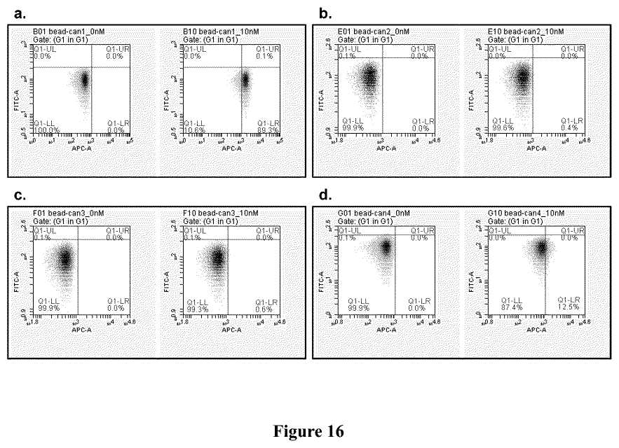

The single-sequenced functionality-conjugated cmFANA aptamers of four of the selected aptamer sequences from the top four families, which all had >100 fold enrichment compared to the starting library, were synthesized, immobilized on beads, and subject to flow cytometry to measure their fluorescence intensity after incubating with 0 nM to 10 nM Alexa Fluor 647 labeled Con A. Of these four, sequence can-1 (80nt) showed the most fluorescence shift (FIG. 15). The bead-based fluorescent binding assay with flow cytometer was also illustrated as contour plots, in which 89.3% of input can-1 aptamer particles bound to Con A at 10 nM, and all four aptamers showed binding when incubating with 10 nM Con A (FIG. 16). This result highlights the promising value of cmFANA as functionality-conjugatable affinity reagent.

### MATERIALS AND METHODS

### General Information

All solvents and reagents for chemical synthesis were purchased from commercial sources and used as supplied. All DNA oligonucleotides were purchased from Integrated DNA Technologies. FANA NTPs (FANA ATP, FANA CTP, FANA GTP, FANA UTP) were obtained from Metkinen Chemistry. Next-generation sequencing was performed using the Amplicon-EZ service provided by Genewiz.

### Experimental Procedure

DNA-Display Cycle for Carbohydrate-Conjugated cmFANA

The DNA-display cycle was adapted from the similar method described in above Example 5. To begin, 10 μL of 10 μM ssDNA Library 1 (N40), 20 μL of 10×NEBuffer 2, 12 μL of 10 μM Hairpin Regenerating Primer, and 150 μL of water was added to a PCR tube to make a master mix of a total volume of 192 μL. The master mix was separated into two PCR tubes with 96 μL master mix in each PCR tube. The PCR tubes were placed in a thermal cycler and heated with an annealing ramp of 95° C. to 45° C. at a rate of 0.1° C. sec⁻¹ to anneal the ssDNA library template-primer. Then, 2 L of 10 mM DNA dNTPs mix and 2 μL of 5 U/μL DNA polymerase I (Klenow) was added into each PCR tube and the reaction mixtures was incubated for 25 min at 25° C. before directly adding 1.5 μL of 20 U/μL Exo I to each tube and further incubated for 30 minutes at 37° C. and then 20 minutes at 80° C. to denature the enzyme. To perform ethanol precipitation, the reaction mixture of the two PCR tubes were combined in a 1.5 mL Eppendorf tube and added nuclease-free water to a total volume of 300 μL, before added with 40 μL of 3 M sodium acetate pH 5.2 and 1 mL of 100% ethanol, followed by freezing at −80° C. for 30 minutes and centrifugation of the frozen stock 30 min at 21,000×g at 4° C. The supernatant was removed and the pellet of dsDNA regeneration product was resuspended with 95 μL water (reserve 5 μL for PAGE analysis) before combining with 90 μL 2×B & W buffer.

To remove the unwanted biotinylated strand, the dsDNA regeneration product solution (180 μL) was first added to 150 μL of MyOne C1 streptavidin beads (supernatant removed and prewashed three times) and mixed on a rotator for 30 minutes at room temperature. The beads were then captured on a magnet and the supernatant was removed. The beads were washed three times with 150 μL 1×B & W before adding 40 μL of 100 mM NaOH and sit for 4 minutes to elute the non-biotinylated ssDNA. The supernatant containing non-biotinylated ssDNA was then transferred to another tube containing 4 μL of 1 M HCl and 1 μL of 1 M Tris-Cl, pH 8.0 for neutralization. The ssDNA was then quantified by NanoDrop Spectrophotometer and stored at −20° C.

cmFANA synthesis by ssDNA hairpin self-extension reaction was performed in 43 μL reaction volumes containing 35 μL of ssDNA library hairpin previously made, 5 μL of 10×Thermopol buffer, and water to a total volume of 43 μL. The ssDNA was self-annealed to its hairpin formation by heating for 5 minutes at 95° C. and slowly cooled down to 4° C. for 10 minutes. The mixture was then added with 1 μL of 10 mM premixed FANA NTPs (FANA ATP, FANA CTP, and FANA GTP only), 1 μL of 10 mM C8-alkyne-FANA UTP (11), and 5 μL of 8 μM lab expressed Tgo polymerase, followed by incubation for 3 hours at 55° C. to perform cmFANA synthesis. The reaction was then added with water to a total volume of 100 μL and treated with ethanol precipitation, in which 10 μL of 3 M sodium acetate (pH 5.2) and 330 μL of 100% ethanol were added to the solution, followed by freezing at −80° C. for 30 minutes and centrifugation of the frozen stock 30 min at 21,000×g at 4° C. The supernatant was removed and the pellet was resuspended with 22 μL PBS buffer 1×, where 2 μL was reserved for PAGE analysis (10% TBE gel).

Click conjugation of hairpin DNA-cmFANA hybrid with Man-N3 was performed by combining the 20 μL hairpin DNA-cmFANA hybrid solution with 30 μL of 100 mM 2-azidoethyl α-(D)-mannopyranoside in DMSO and 40 μL of 20 mM sodium phosphate buffer (pH 8), followed by addition of 20 μL of a 10 mM, premixed solution of Cu:TBTA=1:1 (prepared with 1 mg CuBr+0.7 mL of 10 mM TBTA in 4:3:1 water:DMSO:tBuOH). For preparing positive control for mock selection experiment, exchange 2-azidoethyl α-(D)-mannopyranoside with Azide-PEG3-biotin. The reaction was incubated for 3 hours at room temperature under nitrogen gas bubbling to displace oxygen in the system. The click reaction mixture was again applied with ethanol precipitation, in which 12 μL of 3 M sodium acetate (pH 5.2) and 400 μL of 100% ethanol were added to the solution, followed by freezing and centrifugation of the frozen stock 30 min at 21,000×g at 4° C. The pellet was resuspended with 22 μL of water and 2 μL was reserved for PAGE analysis (10% TBE gel).

Strand displacement of the self-annealed hairpin was performed by combining 20 μL of the hairpin with 6 μL of 10 μM DNA primer H-FP, 5 μL of 10×Thermopol buffer, and 16 μL of water. After heating for 15 seconds at 65° C. in the thermal cycler, the mixture was quickly added with 1 μL of 10 mM DNA dNTP mix and 2 μL of 8 U/μl Bst2.0 WarmStart DNA Polymerase to a total reaction volume of 50 μL, and the reaction was continued to incubate at 65° C. for another 5 minutes, followed by 60° C. for 5 minutes and then hold at 4° C. Ethanol precipitation (add water to 100 μL, add 10 μL of 3 M sodium acetate pH 5.2 and 330 μL of 100% ethanol, freeze, and centrifuge for 30 min at 21,000×g at 4° C.) was performed after the reaction. The pellet was resuspended with 80 μL of water and the mixture was stored at −80° C. To confirm strand displacement result, 8 μL of the strand displacement solution was added with 1 μL exonuclease I (Exo I) buffer, and 1 μL of 20 U/μl Exo I, and then Incubate for 30 min at 37° C. and reserve on ice for PAGE analysis. Both strand displacement samples before and after exonuclease digestion should be analyzed by PAGE (10% TBE gel) to confirm the success of strand displacement.

PCR amplification of the dsDNA was performed by combining the 1 μL strand displacement product with 2.5 μL of 10 μM H-FP-Bio, 2.5 μL of 10 μM H-RP, 10 μL of 5×Q5 reaction buffer, 1 μL of 10 mM DNA dNTPs mix, 1 μL of 2 U/μL Q5 High-Fidelity DNA Polymerase, and 32 μL of water. PCR conditions were as follow: 98° C. for 30 second; 12 cycles of 98° C. for 10 second, 60° C. for 25 second, 72° C. for 26 second; 72° C. for 2 minutes, and hold at 4° C. To remove excess primers and denature the enzymes, 1 μL of 20 U/μl exonuclease I (Exo I) was directly added to the reactions and incubate in a thermal cycler for 30 minutes at 37° C. and then 20 minutes at 80° C. The four reaction mixtures were combined. Ethanol precipitation (add water to 100 μL, add 10 μL of 3 M sodium acetate pH 5.2 and 330 μL of 100% ethanol, freeze, and centrifuge for 30 min at 21,000×g at 4° C.) was again performed. The pellet of PCR product was resuspended into 180 μL 1×B & W buffer and ready for the next step.

To remove unwanted biotinylated strand, the PCR product solution was first added to 150 μL of MyOne C1 streptavidin beads (supernatant removed and prewashed three times) and mixed on a rotator for 30 minutes at room temperature. The beads were then capture on magnet and the supernatant was removed. The beads were washed three times with 150 μL 1×B & W before adding L of 100 mM NaOH and sit for 4 minutes to elute the non-biotinylated ssDNA. The supernatant containing non-biotinylated ssDNA was then transferred to another tube containing 4 μL of 1 M HCl and 1 μL of 1 M Tris-Cl, pH 8.0 for neutralization. The ssDNA was then quantified by NanoDrop Spectrophotometer and stored at −20° C.

Finally, to regenerate the hairpin sequence to give (N+1)th-generation, the 45 μL of ssDNA product was added with 10 μL of 10×NEBuffer 2, 6 μL of 10 μM Hairpin Regenerating Primer, and 35 μL of water. The ssDNA-primer was annealed in thermal cycler with an annealing ramp of 95° C. to 45° C. at a rate of 0.1° C./sec. Then, 2 μL of 10 mM DNA dNTPs mix and 2 μL of 5 U/μl DNA polymerase I (Klenow) was added and the reaction mixture was incubated for 15 min at 25° C. before directly adding 1.5 μL of 20 U/μl Exo I and further incubated for 30 minutes at 37° C. and then 20 minutes at 80° C. Ethanol precipitation (30 μL of 3 M sodium acetate pH 5.2 and 900 μL of 100% ethanol, freeze, and centrifuge for 30 min at 21,000×g at 4° C.) was again performed. The pellet of dsDNA regeneration product was resuspended into 180 μL 1×B & W buffer. Bead separation was again practiced with exact same procedure described in the previous step to remove unwanted biotinylated strand. The ssDNA hairpin of (N+1)th-generation was quantified by NanoDrop Spectrophotometer and stored at −20° C.

In Vitro Selection with ConA Protein as Target

To begin, 10 μL of 100 μM ssDNA Library 1 (N40), 20 μL of 10×NEBuffer 2, 12 μL of 100 μM Hairpin Regenerating Primer, and 150 μL of water was added to a PCR tube to make a master mix of a total volume of 192 μL. The master mix was separated into two PCR tubes with 96 μL master mix in each PCR tube. The PCR tubes were placed in a thermal cycler and heated with an annealing ramp of 95° C. to 45° C. at a rate of 0.1° C./see to anneal the ssDNA library template-primer. Then, 2 μL of 100 mM DNA dNTPs mix and 2 μL of 5 U/μL DNA polymerase I (Klenow) was added into each PCR tube and the reaction mixtures was incubated for 40 min at 25° C. before directly adding 2 μL of 20 U/μL Exo I to each tube and further incubated for 30 minutes at 37° C. and then 20 minutes at 80° C. to denature the enzyme. To perform ethanol precipitation, the reaction mixture of the two PCR tubes were combined in a 1.5 mL Eppendorf tube and added nuclease-free water to a total volume of 300 μL, before added with 40 μL of 3 M sodium acetate pH 5.2 and 1 mL of 100% ethanol, followed by freezing at −80° C. for 30 minutes and centrifugation of the frozen stock 30 min at 21,000×g at 4° C. The supernatant was removed, and the pellet of dsDNA regeneration product was resuspended with 105 μL water (reserve 5 μL for PAGE analysis) before combining with 100 μL 2×B & W buffer.

To remove unwanted biotinylated strand, the dsDNA regeneration product solution (200 μL) was first added to 200 μL of MyOne C1 streptavidin beads (supernatant removed and prewashed three times with 200 μL 1×B & W buffer) and mixed on a rotator for 30 minutes at room temperature. The beads were then capture on magnet and the supernatant was removed. The beads were washed three times with 200 μL 1×B & W before adding 80 μL of 100 mM NaOH and sit for 4 minutes to elute the non-biotinylated ssDNA. The supernatant containing non-biotinylated ssDNA was then transferred to another tube containing 8 μL of 1 M HCl and 2 μL of 1 M Tris-Cl, pH 8.0 for neutralization. The ssDNA (90 μL total) was then quantified by NanoDrop Spectrophotometer and stored at −20° C.

cmFANA synthesis by ssDNA hairpin self-extension reaction was performed in 50 μL reaction volumes containing 30 μL of ssDNA library hairpin previously made, 5 μL of 10×Thermopol buffer, and water to a total volume of 43 μL. The ssDNA was self-annealed to its hairpin formation by heating for 5 minutes at 95° C. and slowly cooled down to 4° C. for 10 minutes. The mixture was then added with 1 μL of 10 mM premixed FANA NTPs (FANA ATP, FANA CTP, and FANA GTP only), 1 μL of 10 mM C8-alkyne-FANA UTP (compound 11), and 5 μL of 8 μM lab expressed Tgo polymerase, followed by incubation for 3 hours at 55° C. to perform cmFANA synthesis. The reaction was then added with water to a total volume of 100 μL and treated with ethanol precipitation, in which 10 μL of 3 M sodium acetate (pH 5.2) and 330 μL of 100% ethanol were added to the solution, followed by freezing at −80° C. for 30 minutes and centrifugation of the frozen stock 30 min at 21,000×g at 4° C. The supernatant was removed, and the pellet was resuspended with 22 μL PBS buffer 1×, where 2 μL was reserved for PAGE analysis (10% TBE gel).

Click conjugation of hairpin DNA-cmFANA hybrid with Man-N3 was performed by combining the 20 μL hairpin DNA-cmFANA hybrid solution with 30 μL of 100 mM 2-azidoethyl α-(D)-mannopyranoside in DMSO and 40 μL of 20 mM sodium phosphate buffer (pH 8), followed by addition of 20 μL of a 10 mM, premixed solution of Cu:TBTA=1:1 (prepared with 1 mg CuBr+0.7 mL of 10 mM TBTA in 4:3:1 water:DMSO:tBuOH). For preparing positive control for mock selection experiment, exchange 2-azidoethyl α-(D)-mannopyranoside with Azide-PEG3-biotin. The reaction was incubated for 3 hours at room temperature under nitrogen gas bubbling to displace oxygen in the system. The click reaction mixture was again applied with ethanol precipitation, in which 12 μL of 3 M sodium acetate (pH 5.2) and 400 μL of 100% ethanol were added to the solution, followed by freezing and centrifugation of the frozen stock 30 min at 21,000×g at 4° C. The pellet was resuspended with 22 μL of water and 2 μL was reserved for PAGE analysis (10% TBE gel).

Strand displacement of the self-annealed hairpin was performed by combining 20 μL of the functionalized double-stranded hairpin product with 6 μL of 10 μM DNA primer H-FP, 5 μL of 10×Thermopol buffer, and 16 μL of water. After heating for 15 seconds at 65° C. in the thermal cycler, the mixture was quickly added with 1 μL of 10 mM DNA dNTP mix and 2 μL of 8 U/μl Bst2.0 WarmStart DNA Polymerase to a total reaction volume of 50 μL, and the reaction was continued to incubate at 65° C. for another 5 minutes, followed by 60° C. for 5 minutes and then hold at 4° C. Ethanol precipitation (add water to 100 μL, add 10 μL of 3 M sodium acetate pH 5.2 and 330 μL of 100% ethanol, freeze, and centrifuge for 30 min at 21,000×g at 4° C.) was performed after the reaction. The pellet was resuspended with 80 μL of water and the mixture was stored at −80° C. To confirm strand displacement result, 8 μL of the strand displacement solution was added with 1 μL exonuclease I (Exo I) buffer, and 1 μL of 20 U/μl Exo I, and then Incubate for 30 min at 37° C. and reserve on ice for PAGE analysis. Both strand displacement samples before and after exonuclease digestion should be analyzed by PAGE (10% TBE gel) to confirm the success of strand displacement.

The selection process starts by adding 39 μL of the prepared strand displacement product into a clean 1.5 mL Eppendorf tube, then add in 5 μL of 10×PBS, 5 μL of 25 mM MgCl2, 0.5 L of 100 mM CaCl2), 0.5 μL of 100 mM MnCl2, and 0.5 μL of 1% Tween 20 diluted in water. To this solution, 5 μL of 570 mM of biotinylated Con A protein in selection buffer (SB; 1×PBS, 2.5 mM MgCl2, 1 mM CaCl2), 0.1 mM MnCl2, 0.01% Tween 20) was added and allowed to incubate at room temperature with rotation for 1 hr. Then, 2.5 μL of MyOne C1 streptavidin beads (supernatant removed and prewashed three times with SB) was added to the mixture, the solution mixture was further diluted with 150 μL SB and mixed on a rotator for 30 minutes at room temperature to recover aptamer-bind biotinylated Con A. The beads were then capture on magnet and the supernatant was removed. The beads were washed ten times with 200 μL SB before resuspending in 50 μL water. To recover the dsDNA genotype, the following exonuclease digestion was prepared:
40 μL of beads (from the above mentioned 50 μL resuspension in water)
5 μL 10×Exo I buffer
2 μL Exo I.
   Incubate 30 min at 37° C. in the thermal cycler and then 20 minutes at 80° C. to denature the enzyme, reserve on ice. The beads were then capture on magnet and the supernatant was collected.

PCR amplification of the dsDNA was performed by combining the 0.5 μL recovered post-selection dsDNA library with 2.5 μL of 10 μM H-FP-Bio, 2.5 μL of 10 μM H-RP, 10 μL of 5×Q5 reaction buffer, 1 μL of 10 mM DNA dNTPs mix, 1 μL of 2 U/μL Q5 High-Fidelity DNA Polymerase, and water to a total volume of 50 μL. PCR conditions were as follow: 98° C. for 30 second; 20 cycles of 98° C. for 10 second, 62° C. for 30 second, 72° C. for 26 second; 72° C. for 2 minutes, and hold at 4° C. To remove excess primers and denature the enzymes, 1 μL of 20 U/μl exonuclease I (Exo I) was directly added to the reactions and incubate in a thermal cycler for 30 minutes at 37° C. and then 20 minutes at 80° C. A total of fifty 50 μL PCR mixtures were prepared and combined in a 15 mL conical centrifuge tube. Ethanol precipitation (add 0.25 mL of 3 M sodium acetate pH 5.2 and 7 mL of 100% ethanol, freeze, and centrifuge for 45 min at 5,000×g at 4° C.) was performed. The pellet was dissolved with 300 μL water, followed by purification using MinElute spin columns. The PCR product was eluted with 100 μL water and was added with 100 μL 2×B & W buffer and ready for the next step.

To remove the unwanted biotinylated strand, the PCR product solution was first added to 200 μL of MyOne C1 streptavidin beads (supernatant removed and prewashed three times) and mixed on a rotator for 30 minutes at room temperature. The beads were then capture on magnet and the supernatant was removed. The beads were washed three times with 200 μL 1×B & W before adding L of 100 mM NaOH and sit for 4 minutes to elute the non-biotinylated ssDNA. The supernatant containing non-biotinylated ssDNA was then transferred to another tube containing 4 μL of 1 M HCl and 1 μL of 1 M Tris-Cl, pH 8.0 for neutralization. The ssDNA selection genotype (94nt) was then quantified by NanoDrop Spectrophotometer and stored at −20° C.

Finally, to regenerate the hairpin sequence to give (N+1)th-generation, the 45 μL of ssDNA product was added with 10 μL of 10×NEBuffer 2, 6 μL of 100 μM Hairpin Regenerating Primer, and 35 μL of water. The ssDNA-primer was annealed in thermal cycler with an annealing ramp of 95° C. to 45° C. at a rate of 0.1° C./sec. Then, 2 μL of 50 mM DNA dNTPs mix and 2 μL of 5 U/μl DNA polymerase I (Klenow) was added and the reaction mixture was incubated for 40 min at 25° C. before directly adding 1.5 μL of 20 U/μl Exo I and further incubated for 30 minutes at 37° C. and then 20 minutes at 80° C. Ethanol precipitation (add water to a total volume of 300 μL, then add 30 μL of 3 M sodium acetate pH 5.2 and 900 μL of 100% ethanol, freeze, and centrifuge for 30 min at 21,000×g at 4° C.) was again performed. The pellet of dsDNA regeneration product was resuspended into 180 μL 1×B & W buffer. Bead separation was again practiced with exact same procedure described in the previous step to remove unwanted biotinylated strand. The ssDNA hairpin of (N+1)th-generation was quantified by NanoDrop Spectrophotometer and stored at −20° C.

A second-generation ssDNA hairpin self-extension reaction was performed as described above and an aliquot of the extended hairpin DNA-cmFANA hybrid product was analyzed by PAGE (10% TBE gel) to confirm the success of the DNA-display cycle.

A second-generation ssDNA hairpin self-extension reaction was performed as described above and an aliquot of the extended hairpin DNA-cmFANA hybrid product was analyzed by PAGE (10% TBE gel) to confirm the success of the DNA-display cycle.

### Next-Generation Sequencing of Enriched Library

The next-generation sequencing of the library was performed according to the Amplicon-EZ workflow provided by Genewiz. Briefly, the ssDNA selection genotype (94nt) of after the first and second rounds of selection were further amplified by PCR with extended forward (Amplicon_FP_XL) and reverse primers (Amplicon_RP_XL) to add extra 56 bp to the dsDNA amplicon sample, making the length of the amplicon sequence 150 bp without the adapters, as well as to install the adapter and a spacer such that the length of the PCR product is 215 bp (dsDNA amplicon sample 2) required for Genewiz Amplicon-EZ sequencing. The sequencing results and FASTQ files were analyzed by a python code that provided the total number of reads, read length, number of unique sequences, and visualization (FIG. 2-6). The original files were also uploaded to FASTAptameR2.0 to generate respective FASTA files and analyze the cluster count, cluster diversity, and cluster visualization (FIG. 2-7) using the online FASTAptameR2.0 features “Count”, “Cluster”, and “Diversity” (the R-based analyzation platform FASTAptameR2.0 can be accessed online at https://fastaptamer2.missouri.edu/).

### Generating Single-Sequenced Mannose-cmFANA Aptamer Candidates

Single-stranded cmFANA were generated by first preparing a master mix solution in a 1.5 mL Eppendorf tube containing 20 μL of 100 μM BindConA_primer_Bio, 20 μL of 100 μM (T_BindConA_can-1, or T_BindConA_can-2, or T_BindConA_can-3, or T_BindConA_can-4), 200 μL of 10×ThermoPol buffer, and 560 μL ultra-pure water (800 μL total). The 1.5 mL Eppendorf tube containing the mast mix was placed on a hot plate with temperature set at 90° C. for 10 minutes and cooling on ice for 15 minutes. The master mix was transfer to a larger tube before adding 200 μL of 1 mM of FANA NTPs (A, C, and G), 200 μL of 1 mM C8-alkyne-FANA UTP (compound 11, above described), 400 μL of lab expressed Tgo polymerase (a final concentration of 1 μM), and water to a total volume of 2 mL. The mixture was separated into four 1.5 mL Eppendorf tubes (500 μL each) and placed on hotplate with temperature set at 55° C. Primer-extension reactions were performed for 3 hours at 55° C. before placing on ice.

The primer extension reaction mixtures were collected in a 15 mL centrifuge tube and added with 0.25 mL of 3 M sodium acetate (pH 5.2) solution and 6.875 mL of 100% ethanol, followed by freezing at −80° C. for 30 minutes or liquid nitrogen for 5 minutes. The frozen stock was then centrifuged for 30 min at 4000×g at 4° C. to precipitate the DNA-cmFANA hybrid. The pellet was dissolved with 300 μL water, followed by purification using MinElute spin columns where the primer extension product was eluted with 90 μL of TE buffer (10 mM Tris-Cl, 1 mM EDTA, pH 8). The collected primer extension product was again applied with ethanol precipitation, in which 20 μL of 3 M sodium acetate (pH 5.2) and 600 μL of 100% ethanol were added to the solution, followed by freezing and centrifugation of the frozen stock 30 min at 21,000×g at 4° C. The precipitated DNA-cmFANA hybrid was resuspended in 20 μL 1×PBS buffer.

To perform click conjugation, the 20 μL DNA-cmFANA hybrid solution was added with 20 μL of 100 mM 2-azidoethyl-α-(D)-mannopyranoside in DMSO and 40 μL 20 mM sodium phosphate buffer (pH 8) in a 1.5 mL Eppendorf tube. The cap of the tube was cut off and sealed with a rubber septum, and the system was purged with nitrogen gas for 15 minutes. The click reaction was initiated by adding 20 μL of a 10 mM, premixed solution of Cu:TBTA=1:1 (prepared with 1 mg CuBr+0.7 mL of 10 mM TBTA in 4:3:1 water:DMSO:tBuOH), followed by nitrogen gas purge for 5 minutes and incubated for 4 hours. The click reaction mixture was then applied with ethanol precipitation, in which 10 μL of 3 M sodium acetate (pH 5.2) and 330 μL of 100% ethanol were added to the solution, followed by freezing and centrifugation of the frozen stock 30 min at 21,000×g at 4° C. The sugar-conjugated DNA-cmFANA hybrid pellet was resuspended with 200 μL 1×B & W buffer (5 mM Tris-HCl pH 7.5, 0.5 mM EDTA, 1 M NaCl) and ready for the next step.

To perform bead separation, 200 μL MyOne C1 streptavidin beads was added to a new 1.5 mL Eppendorf tube. The beads were captured on the side of the tube with a magnet and the supernatant was removed. The beads were washed three times with 200 μL 1×B & W. The click product solution was added to the beads and mixed on a rotator for 30 minutes at room temperature. Then, the beads were captured, and the supernatant was removed. The beads with sugar-conjugated DNA-cmFANA hybrid were washed three times with 200 μL 1×B & W, and then treated twice with 50 μL of freshly-prepared 0.25 M NaOH solution to remove non-biotinylated strand and generate single-stranded sugar-cmFANA on the beads. The beads were again captured, and the supernatant was removed. Finally, the beads with cmFANA aptamer were washed again with 1 mL 1×PBS buffer before resuspending in 100 μL 1×PBS buffer.

### General Procedure for Bead-Based Binding Assay for Fluorescently Labeled ConA

Around 7-10×106 bead particles (biotinylated functionalized aptamer immobilized on Dynabeads MyOne Streptavidin C1) were incubated with varying concentrations of fluorescently labeled Con A (0-10 nM) in SB for 1 hour on a rotator at room temperature. The beads were washed three times with 200 μL 1×BD Sheath Solution after incubation and resuspended in 10 μL 1×BD Sheath Solution. From this, 3 μL of the beads were transferred to 300 μL 1×BD Sheath Solution for flow cytometry. The beads were analyzed using the BD Accuri C6 flow cytometer, and the mean fluorescence and/or percentage of bound particles were measured in the relevant fluorescence channel(s).

TABLE 2
DNA Sequences

| SEQ. | | |
|---|---|---|
| ID. | | |
| NO. | Name | DNA sequence |
| | In vitro Selection | |
| 07 | Library 1 (N40)(94 nt) | 5′-CC TAT AGC CGT TTG CAC AAG |
| | | (N1:25 25 25 25)(N1)(N1)(N1)(N1)(N1) |
| | | (N1)(N1)(N1)(N1)(N1)(N1)(N1)(N1)(N1) |
| | | (N1)(N1)(N1)(N1)(N1)(N1)(N1)(N1)(N1) |
| | | (N1)(N1)(N1)(N1)(N1)(N1)(N1)(N1)(N1) |
| | | (N1)(N1)(N1)(N1)(N1)(N1)(N1) CCC |
| | | GTA CCC GTT AAG ATT ACT TCG GAC |
| | | TGG GAT C-3′ |
| 08 | H-FP (20 nt) | 5′-GAT CCC AGT CCG AAG TAA TC-3′ |
| 09 | H-RP (20 nt) | 5′-CCT ATA GCC GTT TGC ACA AG-3′ |
| 10 | H-FP-Bio (20 nt) | 5′-/5BiotinTEG/GAT CCC AGT CCG |
| | | AAG TAA TC-3′ |
| 11 | Hairpin Regenerating | 5′-/5BiotinTEG/CCC GTA CCC GAA |
| | Primer (60 nt) | TAT AAA ATA AAA ATA TAA AAT ATA |
| | | AAA T GA TCC CAG TCC GAA GTA ATC-3′ |
| | Next-generation sequencing for in vitro selection | |
| 12 | ssDNA selection genotype | 5′-CC TAT AGC CGT TTG CAC AAG |
| | (N40)(94 nt) | (N1:25 25 25 25)(N1)(N1)(N1)(N1)(N1) |
| | | (N1)(N1)(N1)(N1)(N1)(N1)(N1)(N1)(N1) |
| | | (N1)(N1)(N1)(N1)(N1)(N1)(N1)(N1)(N1) |
| | | (N1)(N1)(N1)(N1)(N1)(N1)(N1)(N1)(N1) |
| | | (N1)(N1)(N1)(N1)(N1)(N1)(N1) CCC |
| | | GTA CCC GTT AAG ATT ACT TCG GAC |
| | | TGG GAT C-3′ |
| 13 | Amplicon_FP_XL | 5′-ACA CTC TTT CCC TAC ACG ACG |
| | | CTC TTC CGA TCT GAT AGC AT TTT |
| | | TTT TTT TTT TTT TTT TT CC TAT AGC |
| | | CGT TTG CAC AAG-3′ |
| 14 | Amplicon_RP_XL | 5′-GAC TGG AGT TCA GAC GTG TGC |
| | | TCT TCC GAT CTG ACT CCT G TTT |
| | | TTT TTT TTT TTT TTT TT GAT CCC |
| | | AGT CCG AAG TAA TC-3′ |
| 15 | dsDNA amplicon sample | 5′-ACA CTC TTT CCC TAC ACG ACG |
| | 2 (215 bp) | CTC TTC CGA TCT GAT AGC AT TTT |
| | | TTT TTT TTT TTT TTT TT CC TAT AGC |
| | | CGT TTG CAC AAG (N1)(N1) |
| | | (N1)(N1)(N1)(N1)(N1)(N1)(N1)(N1)(N1) |
| | | (N1)(N1)(N1)(N1)(N1)(N1)(N1)(N1)(N1) |
| | | (N1)(N1)(N1)(N1)(N1)(N1)(N1)(N1)(N1) |
| | | (N1)(N1)(N1)(N1)(N1)(N1)(N1)(N1)(N1) |
| | | (N1) CC CGT ACC CG T TAA GAT TAC |
| | | TTC GGA CTG GGA TC A AAA AAA |
| | | AAA AAA AAA AAA A CA GGA GTC |
| | | AGA TCG GAA GAG CAC ACG TCT |
| | | GAA CTC CAG TC-3′ |
| | Generation of single-stranded cmFANA conjugates for binding assay | |
| 16 | BindConA_primer_Bio | 5′-/5BiotinTEG/ATT TTA TAT TCG GGT |
| | | ACG GG-3′ |
| 17 | T_BindConA_can-1 | 5′-CCT ATA GCC GTT TGC ACA AG T |
| | | CGA GAT CTT AGG CAT CTT TGA CGA |
| | | TGA CTT CGT CTA ACA CCC GTA CCC |
| | | GAA TAT AAA AT AAC AAC AAC AAC |
| | | AAC AAC AA-3′ |
| 18 | T_BindConA_can-2 | 5′-CCT ATA GCC GTT TGC ACA AGT |
| | | ACG ACC CCA ATC CAT CGT AGA ACT |
| | | ATC CCG AAT CCT TGT CCC GTA CCC |
| | | GAA TAT AAA AT AAC AAC AAC AAC |
| | | AAC AAC AA-3′ |
| 19 | T_BindConA_can-3 | 5′-CCT ATA GCC GTT TGC ACA AG G |
| | | TCA AAG GCA CGT GTT GAA ACC ATA |
| | | TTA CTA AGG CAC ACG CCC GTA CCC |
| | | GAA TAT AAA AT AAC AAC AAC AAC |
| | | AAC AAC AA-3′ |
| 20 | T_BindConA_can-4 | 5′-CCT ATA GCC GTT TGC ACA AGA |
| | | GTG ATA TGC TCA GAA ATA TCA TTC |
| | | TGG TCA TAA ATA AAT CCC GTA CCC |
| | | GAA TAT AAA AT AAC AAC AAC AAC |
| | | AAC AAC AA-3′ |
| 21 | T_can-1_scrambled | 5′-CCT ATA GCC GTT TGC ACA AG C |
| | | CTT ATA TTT GCC TCA GTG TCA ATA |
| | | ACT TAA TGA GGG GCC CCC GTA CCC |
| | | GAA TAT AAA AT AAC AAC AAC AAC |
| | | AAC AAC AA-3′ |
| 22 | ssDNA-cmFANA hybrid | 5′-/5BiotinTEG/ATT TTA TAT TCG GGT |
| | product can-1 (80 nt) | ACG GG U*GU* U*AG ACG AAG U*CA |
| | | U*CG U*CA AAG AU*G CCU* AAG AU*C |
| | | U*CG A C*U* GU*G CAA ACG GC |
| | | U*AU*A GG-3′ |
| 23 | ssDNA-cmFANA hybrid | 5′-/5BiotinTEG/ATT TTA TAT TCG GGT |
| | product can-2 (80 nt) | ACG GG ACA AGG AU*U* CGG GAU* |
| | | AGU* U*CU* ACG AU*G GAU* U*GG |
| | | GGU* CGU* A CU*U* GU*G CAA ACG |
| | | GCU* AU*A GG-3′ |
| 24 | ssDNA-cmFANA hybrid | 5′-/5BiotinTEG/ATT TTA TAT TCG GGT |
| | product can-3 (80 nt) | ACG GG CGU* GU*G CCU* U*AG U*AA |
| | | U*AU* GGU* U*U*C AAC ACG U*GC |
| | | CU*U* U*GA C C*U* G UNG CAA ACG |
| | | GCU* AU*A GG-3′ |
| 25 | ssDNA-cmFANA hybrid | 5′-/5BiotinTEG/ATT TTA TAT TCG GGT |
| | product can-4 (80 nt) | ACG GG AU*U* U*AU* U*U*A U*GA |
| | | CCA GAA U*GA U*AU* U*U*C U*GA |
| | | GCA U*AU* CAC U* CU* U*GU*G CAA |
| | | ACG GCU* AU*A GG-3′ |
| 26 | ssDNA-cmFANA hybrid | 5′-/5BiotinTEG/ATT TTA TAT TCG GGT |
| | product can-1-scrambled | ACG GG GGC CCC U*CA U* U*A AG U* |
| | (80 nt) | U*A U* U*GA CAC U GA GGC AAA U*A |
| | | U* AAG G CU*U* GU*G CAA ACG GC |
| | | U*AU*A GG-3′ |

It is understood that, while particular embodiments have been illustrated and described, various modifications may be made thereto and are contemplated herein. It is also understood that the disclosure is not limited by the specific examples provided herein. The description and illustration of embodiments and examples of the disclosure herein are not intended to be construed in a limiting sense. It is further understood that all aspects of the disclosure are not limited to the specific depictions, configurations or relative proportions set forth herein, which may depend upon a variety of conditions and variables. Various modifications and variations in form and detail of the embodiments and examples of the disclosure will be apparent to a person skilled in the art. It is therefore contemplated that the disclosure also covers any and all such modifications, variations and equivalents.

What is claimed is:

1. A nucleotide compound designated 5-octa-1,7-diynyluracil 2′-deoxy-2′-fluoroarabino having the following structure, or a salt, solvate or hydrate thereof:

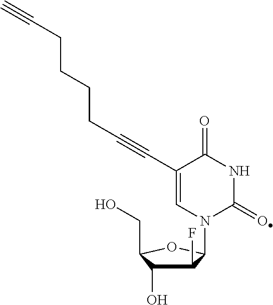

2. A method of synthesizing the compound of claim 1 as described in FIG. 2a.

3. A compound designated 5-octa-1,7-diynyluracil 2′-deoxy-2′-fluoroarabino triphosphate and having the structure: 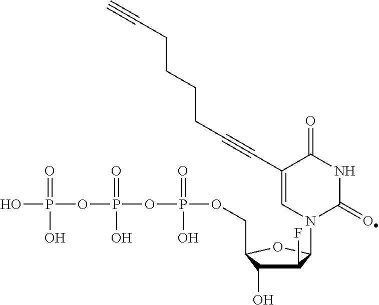
or a salt, solvate or hydrate thereof.

4. A method of synthesizing the compound of claim 3 as described in FIG. 2b.

5. A method of synthesizing an aptamer, comprising mixing a nucleic acid template, a primer, 5-octa-1,7-diynyluracil 2′-deoxy-2′-fluoroarabino triphosphate, and a wild-type or engineered DNA polymerase.

6. The method of claim 5, wherein the DNA polymerase is Thermococcus gorgonarius [Tgo] DNA polymerase.

7. The method of claim 5, further comprising mixing 2′-deoxy-2′-fluoroarabino nucleic acid (FANA) analogs of adenosine-5′-triphosphate (ATP), cytidine-5′-triphosphate (CTP) and guanosine-5′-triphosphate (GTP).

8. The method of synthesizing an aptamer according to claim 5, wherein the primer is labeled with an agent that promotes isolation, separation or detection of a single-stranded or double-stranded aptamer comprising one or more 5-octa-1,7-diynyluracil 2′-deoxy-2′-fluoroarabino nucleotides.

9. A method of conjugating a single-stranded or double-stranded aptamer comprising one or more 5-octa-1,7-diynyluracil 2′-deoxy-2′-fluoroarabino nucleotides to an azide-functionalized compound via a copper(I)-catalyzed azide-alkyne cycloaddition (CuAAC) reaction.

10. A functionality-conjugated click handle-modified 2′-deoxy-2′-fluoroarabino nucleic acid (cmFANA) aptamer comprising a 5-octa-1,7-diynyluracil 2′-deoxy-2′-fluoroarabino monophosphate.

11. The functionality-conjugated cmFANA aptamer of claim 10 wherein the aptamer sequence is one of SEQ ID NOs: 1-6.

12. The functionality-conjugated cmFANA aptamer of claim 10 wherein the functionality-conjugation is an azide functionalization.

13. The functionality-conjugated cmFANA aptamer of claim 10 wherein the functionality is at least one selected from the group of: carbohydrates, monosaccharides, oligosaccharides, fluorophores, and hydrophobic or charged moieties.

14. The functionality-conjugated cmFANA aptamer of claim 10 wherein the functionalization occurs post-polymerization of the aptamer.

15. The functionality-conjugated cmFANA aptamer of claim 14 wherein the functionalization occurs via a copper(I)-catalyzed azid-alkyne cycloaddition (CuAAC) reaction.


SEQUENCE LISTING (26 sequences)

SEQ ID NO: 1 - DNA, 40 bp, synthetic construct
  source 1..40: mol_type = other DNA
tcgagatcttaggcatctttgacgatgacttcgtctaaca

SEQ ID NO: 2 - DNA, 40 bp, synthetic construct
  source 1..40: mol_type = other DNA
tacgaccccaatccatcgtagaactatcccgaatccttgt

SEQ ID NO: 3 - DNA, 40 bp, synthetic construct
  source 1..40: mol_type = other DNA
gtcaaaggcacgtgttgaaaccatattactaaggcacacg

SEQ ID NO: 4 - DNA, 40 bp, synthetic construct
  source 1..40: mol_type = other DNA
agtgatatgctcagaaatatccttctggtcataaataaat

SEQ ID NO: 5 - DNA, 40 bp, synthetic construct
  source 1..40: mol_type = other DNA
atacccaagacctacattatagcagcgacgataattaatg

SEQ ID NO: 6 - DNA, 40 bp, synthetic construct
  source 1..40: mol_type = other DNA
caaaattttcagttcgaccgttcaacttcaactacaaaca

SEQ ID NO: 7 - DNA, 94 bp, synthetic construct
  source 1..94: mol_type = other DNA
cctatagccgtttgcacaagnnnnnnnnnnnnnnnnnnnnnnnnnnnnnnnnnnnnnnnncccgtacccgttaagattacttcggactgggatc

SEQ ID NO: 8 - DNA, 20 bp, synthetic construct
  source 1..20: mol_type = other DNA
gatcccagtccgaagtaatc

SEQ ID NO: 9 - DNA, 20 bp, synthetic construct
  source 1..20: mol_type = other DNA
cctatagccgtttgcacaag

SEQ ID NO: 10 - DNA, 20 bp, synthetic construct
  source 1..20: mol_type = other DNA
  unsure 1: note = /5BiotinTEG/
gatcccagtccgaagtaatc

SEQ ID NO: 11 - DNA, 60 bp, synthetic construct
  source 1..60: mol_type = other DNA
  unsure 1: note = /5BiotinTEG/
cccgtacccgaatataaaataaaaatataaaatataaaatgatcccagtccgaagtaatc

SEQ ID NO: 12 - DNA, 94 bp, synthetic construct; identical to SEQ ID NO: 7
  source 1..94: mol_type = other DNA

SEQ ID NO: 13 - DNA, 81 bp, synthetic construct
  source 1..81: mol_type = other DNA
acactctttccctacacgacgctcttccgatctgatagcatttttttttttttttttttttcctatagccgtttgcacaag

SEQ ID NO: 14 - DNA, 80 bp, synthetic construct
  source 1..80: mol_type = other DNA
gactggagttcagacgtgtgctcttccgatctgactcctgttttttttttttttttttttgatcccagtccgaagtaatc

SEQ ID NO: 15 - DNA, 214 bp, synthetic construct
  source 1..214: mol_type = other DNA
acactctttccctacacgacgctcttccgatctgatagcatttttttttttttttttttttcctatagccgtttgcacaagnnnnnnnnnnnnnnnnnnnnnnnnnnnnnnnnnnnnnnncccgtacccgttaagattacttcggactgggatcaaaaaaaaaaaaaaaaaaaacaggagtcagatcggaagagcacacgtctgaactccagtc

SEQ ID NO: 16 - DNA, 20 bp, synthetic construct
  source 1..20: mol_type = other DNA
  unsure 1: note = /5BiotinTEG/
attttatattcgggtacggg

SEQ ID NO: 17 - DNA, 100 bp, synthetic construct
  source 1..100: mol_type = other DNA
cctatagccgtttgcacaagtcgagatcttaggcatctttgacgatgacttcgtctaacacccgtacccgaatataaaataacaacaacaacaacaacaa

SEQ ID NO: 18 - DNA, 100 bp, synthetic construct
  source 1..100: mol_type = other DNA
cctatagccgtttgcacaagtacgaccccaatccatcgtagaactatcccgaatccttgtcccgtacccgaatataaaataacaacaacaacaacaacaa

SEQ ID NO: 19 - DNA, 100 bp, synthetic construct
  source 1..100: mol_type = other DNA
cctatagccgtttgcacaaggtcaaaggcacgtgttgaaaccatattactaaggcacacgcccgtacccgaatataaaataacaacaacaacaacaacaa

SEQ ID NO: 20 - DNA, 100 bp, synthetic construct
  source 1..100: mol_type = other DNA
cctatagccgtttgcacaagagtgatatgctcagaaatatcattctggtcataaataaatcccgtacccgaatataaaataacaacaacaacaacaacaa

SEQ ID NO: 21 - DNA, 100 bp, synthetic construct
  source 1..100: mol_type = other DNA
cctatagccgtttgcacaagccttatatttgcctcagtgtcaataacttaatgaggggcccccgtacccgaatataaaataacaacaacaacaacaacaa

SEQ ID NO: 22 - DNA, 80 bp, synthetic construct
  source 1..80: mol_type = other DNA
  unsure 1: note = /5BiotinTEG/
attttatattcgggtacgggtgttagacgaagtcatcgtcaaagatgcctaagatctcgacttgtgcaaacggctatagg

SEQ ID NO: 23 - DNA, 80 bp, synthetic construct
  source 1..80: mol_type = other DNA
  unsure 1: note = /5BiotinTEG/
attttatattcgggtacgggacaaggattcgggatagttctacgatggattggggtcgtacttgtgcaaacggctatagg

SEQ ID NO: 24 - DNA, 80 bp, synthetic construct
  source 1..80: mol_type = other DNA
  unsure 1: note = /5BiotinTEG/
attttatattcgggtacgggcgtgtgccttagtaatatggtttcaacacgtgcctttgaccttgtgcaaacggctatagg

SEQ ID NO: 25 - DNA, 80 bp, synthetic construct
  source 1..80: mol_type = other DNA
  unsure 1: note = /5BiotinTEG/
attttatattcgggtacgggatttatttatgaccagaatgatatttctgagcatatcactcttgtgcaaacggctatagg

SEQ ID NO: 26 - DNA, 80 bp, synthetic construct
  source 1..80: mol_type = other DNA
  unsure 1: note = /5BiotinTEG/
attttatattcgggtacgggggcccctcattaagttattgacactgaggcaaatataaggcttgtgcaaacggctatagg